(19) United States
(12) Patent Application Publication
(10) Pub. No.: US 2026/0000664 A1
(43) Pub. Date: Jan. 1, 2026

(54) TREATING PATIENTS HARBORING AN ISOCITRATE DEHYDROGENASE-1 (IDH-1) MUTATION

(71) Applicant: FORMA Therapeutics, Inc., Watertown, MA (US)

(72) Inventors: Patrick F. Kelly, Concord, MA (US); Alan Collis, Lexington, MA (US); Jeff Davis, Hingham, MA (US); Duncan Walker, Boulder, CO (US); Susan Ashwell, Carlisle, MA (US); Blythe Thomson, Cincinnati, OH (US); Wei Lu, Newton, MA (US)

(73) Assignee: FORMA Therapeutics, Inc., Watertown, MA (US)

(21) Appl. No.: 19/041,868

(22) Filed: Jan. 30, 2025

Related U.S. Application Data
(63) Continuation of application No. 17/986,414, filed on Nov. 14, 2022, now abandoned.
Application No. 17/986,414 is a continuation of application No. 17/112,269, filed on Dec. 4, 2020, now Pat. No. 11,497,743.
Application No. 17/112,269 is a continuation of application No. 16/526,593, filed on Jul. 30, 2019, now Pat. No. 11,013,734.
Application No. 16/526,593 is a continuation-in-part of application No. 16/431,588, filed on Jun. 4, 2019, now Pat. No. 11,013,733.
Application No. 16/431,588 is a continuation-in-part of application No. PCT/US19/32742, filed on May 16, 2019.
Application No. 16/431,588 is a continuation-in-part of application No. PCT/US19/32747, filed on May 16, 2019.
Application No. 16/431,588 is a continuation-in-part of application No. 16/414,505, filed on May 16, 2019, now Pat. No. 11,576,906.
Application No. 16/414,505 is a continuation-in-part of application No. 16/414,716, filed on May 16, 2019, now Pat. No. 10,532,047.
Application No. 16/526,593 is a continuation-in-part of application No. PCT/US19/32747, filed on May 16, 2019.
Application No. PCT/US19/32747 is a continuation-in-part of application No. 16/414,505, filed on May 16, 2019, now Pat. No. 11,576,906.
(60) Provisional application No. 62/672,462, filed on May 16, 2018.
(60) Provisional application No. 62/672,461, filed on May 16, 2018.
(60) Provisional application No. 62/672,462, filed on May 16, 2018.
(60) Provisional application No. 62/672,461, filed on May 16, 2018.
(60) Provisional application No. 62/672,462, filed on May 16, 2018.
(60) Provisional application No. 62/672,461, filed on May 16, 2018.
(60) Provisional application No. 62/672,462, filed on May 16, 2018.
(60) Provisional application No. 62/672,461, filed on May 16, 2018.
(60) Provisional application No. 62/680,566, filed on Jun. 4, 2018.
(60) Provisional application No. 62/680,571, filed on Jun. 4, 2018.
(60) Provisional application No. 62/680,560, filed on Jun. 4, 2018.
(60) Provisional application No. 62/680,562, filed on Jun. 4, 2018.
(60) Provisional application No. 62/680,566, filed on Jun. 4, 2018.
(60) Provisional application No. 62/680,571, filed on Jun. 4, 2018.
(60) Provisional application No. 62/680,560, filed on Jun. 4, 2018.
(60) Provisional application No. 62/680,562, filed on Jun. 4, 2018.
(60) Provisional application No. 62/692,591, filed on Jun. 29, 2018.
(60) Provisional application No. 62/692,591, filed on Jun. 29, 2018.
(60) Provisional application No. 62/692,598, filed on Jun. 29, 2018.
(60) Provisional application No. 62/692,601, filed on Jun. 29, 2018.
(60) Provisional application No. 62/692,604, filed on Jun. 29, 2018.
(60) Provisional application No. 62/692,605, filed on Jun. 29, 2018.
(60) Provisional application No. 62/692,591, filed on Jun. 29, 2018.
(60) Provisional application No. 62/692,598, filed on Jun. 29, 2018.
(60) Provisional application No. 62/692,601, filed on Jun. 29, 2018.
(60) Provisional application No. 62/692,604, filed on Jun. 29, 2018.
(60) Provisional application No. 62/692,605, filed on Jun. 29, 2018.
(60) Provisional application No. 62/692,591, filed on Jun. 29, 2018.
(60) Provisional application No. 62/701,487, filed on Jul. 20, 2018.
(60) Provisional application No. 62/712,160, filed on Jul. 30, 2018.
(60) Provisional application No. 62/712,160, filed on Jul. 30, 2018.
(60) Provisional application No. 62/773,562, filed on Nov. 30, 2018.
(60) Provisional application No. 62/773,562, filed on Nov. 30, 2018.
(60) Provisional application No. 62/798,677, filed on Jan. 30, 2019.
(60) Provisional application No. 62/798,681, filed on Jan. 30, 2019.
(60) Provisional application No. 62/798,684, filed on Jan. 30, 2019.
(60) Provisional application No. 62/798,687, filed on Jan. 30, 2019.
(60) Provisional application No. 62/798,690, filed on Jan. 30, 2019.
(60) Provisional application No. 62/798,677, filed on Jan. 30, 2019.
(60) Provisional application No. 62/798,681, filed on Jan. 30, 2019.
(60) Provisional application No. 62/798,684, filed on Jan. 30, 2019.
(60) Provisional application No. 62/798,687, filed on Jan. 30, 2019.
(60) Provisional application No. 62/798,690, filed on Jan. 30, 2019.
(60) Provisional application No. 62/812,367, filed on Mar. 1, 2019.
(60) Provisional application No. 62/812,367, filed on Mar. 1, 2019.

(51) Int. Cl. A61K 31/4709; A61P 35/02; C12Q 1/686; C12Q 1/6886

(52) U.S. Cl. CPC A61K 31/4709; A61P 35/02; C12Q 1/686; C12Q 1/6886; C12Q 2531/113; C12Q 2561/113; C12Q 2600/156

(57) ABSTRACT

Methods of treating patients diagnosed with AML or MDS harboring mutant IDH-1 include detecting an IDH1 mutation and the therapeutic administration of an inhibitor of a mutant IDH-1 as a single agent, or in combination with azacitidine (AZA) or cytarabine.

### CROSS REFERENCE TO RELATED APPLICATIONS

This application is a continuation of U.S. application Ser. No. 17/112,269, filed Dec. 4, 2020, which is a continuation of U.S. application Ser. No. 16/526,593, filed Jul. 30, 2019, now U.S. Pat. No. 11,013,734, which claims the benefit of and priority to U.S. Provisional Application No. 62/712,160, filed Jul. 30, 2018, which is incorporated by reference herein in its entirety; and
said 16/526,593 application is a continuation-in-part of U.S. application Ser. No. 16/414,505, filed May 16, 2019, and International Application No. PCT/US19/32747, filed May 16, 2019, each of which claims the benefit of and priority to U.S. Provisional Application No. 62/672,461, filed May 16, 2018; U.S. Provisional Application No. 62/672,462, filed May 16, 2018; U.S. Provisional Application No. 62/680,566, filed Jun. 4, 2018; U.S. Provisional Application No. 62/680,571, filed Jun. 4, 2018; U.S. Provisional Application No. 62/680,560, filed Jun. 4, 2018; U.S. Provisional Application No. 62/680,562, filed Jun. 4, 2018; U.S. Provisional Application No. 62/692,598, filed Jun. 29, 2018; U.S. Provisional Application No. 62/692,601, filed Jun. 29, 2018; U.S. Provisional Application No. 62/692,604, filed Jun. 29, 2018; U.S. Provisional Application No. 62/692,605, filed Jun. 29, 2018; U.S. Provisional Application No. 62/692,591, filed Jun. 29, 2018; U.S. Provisional Application No. 62/773,562, filed Nov. 30, 2018; U.S. Provisional Application No. 62/798,677, filed Jan. 30, 2019; U.S. Provisional Application No. 62/798,681, filed Jan. 30, 2019; U.S. Provisional Application No. 62/798,684, filed Jan. 30, 2019; U.S. Provisional Application No. 62/798,687, filed Jan. 30, 2019; U.S. Provisional Application No. 62/798,690, filed Jan. 30, 2019; and U.S. Provisional Application No. 62/812,367, filed Mar. 1, 2019; and
said 16/526,593 is a continuation-in-part of U.S. application Ser. No. 16/431,588, filed Jun. 4, 2019, now U.S. Pat. No. 11,013,733, which claims the benefit of and priority to U.S. Provisional Application No. 62/701,487, filed Jul. 20, 2018; and U.S. Provisional Application No. U.S. 62/712,160, filed Jul. 30, 2018; and
is a continuation-in-part of U.S. application Ser. No. 16/414,505, filed May 16, 2019, and International Application No. PCT/US19/32747, filed May 16, 2019, each of which claims the benefit of and priority to U.S. Provisional Application No. 62/672,461, filed May 16, 2018; U.S. Provisional Application No. 62/672,462, filed May 16, 2018; U.S. Provisional Application No. 62/680,566, filed Jun. 4, 2018; U.S. Provisional Application No. 62/680,571, filed Jun. 4, 2018; U.S. Provisional Application No. 62/680,560, filed Jun. 4, 2018; U.S. Provisional Application No. 62/680,562, filed Jun. 4, 2018; U.S. Provisional Application No. 62/692,598, filed Jun. 29, 2018; U.S. Provisional Application No. 62/692,601, filed Jun. 29, 2018; U.S. Provisional Application No. 62/692,604, filed Jun. 29, 2018; U.S. Provisional Application No. 62/692,605, filed Jun. 29, 2018; U.S. Provisional Application No. 62/692,591, filed Jun. 29, 2018; U.S. Provisional Application No. 62/773,562, filed Nov. 30, 2018; U.S. Provisional Application No. 62/798,677, filed Jan. 30, 2019; U.S. Provisional Application No. 62/798,681, filed Jan. 30, 2019; U.S. Provisional Application No. 62/798,684, filed Jan. 30, 2019; U.S. Provisional Application No. 62/798,687, filed Jan. 30, 2019; U.S. Provisional Application No. 62/798,690, filed Jan. 30, 2019; and U.S. Provisional Application No. 62/812,367, filed Mar. 1, 2019;
and is a continuation-in-part of U.S. application Ser. No. 16/414,716, filed May 16, 2019, now U.S. Pat. No. 10,532,047, and International Application No. PCT/US19/32742, filed May 16, 2019, each of which claims the benefit of and priority to U.S. Provisional Application No. 62/672,461, filed on May 16, 2018; U.S. Provisional Application No. 62/672,462, filed on May 16, 2018; and U.S. Provisional Application No. 62/692,591, filed on Jun. 29, 2018;
the contents of each of the applications listed above are hereby incorporated by reference in their entirety.

### TECHNICAL FIELD

This disclosure relates to the treatment of acute myeloid leukemia (AML) or myelodysplastic syndrome (MDS). For example, the disclosure provides methods of treating AML or MDS in patients diagnosed as harboring certain mutated forms of isocitrate dehydrogenase 1 (mIDH1 or mt-IDH1).

### BACKGROUND

The metabolic enzyme isocitrate dehydrogenase (IDH1) catalyzes the oxidative decarboxylation of isocitrate to α-ketoglutarate (α-KG). In both hematologic and solid tumor malignancies, mutated IDH1 acquires the neomorphic activity of converting α-KG to 2-hydroxyglutarate (2-HG) and thereby leads to the aberrant accumulation of 2-HG. 2-HG has been proposed to act as an “oncometabolite” that has pleotropic effects on tumorigenesis. Excess production of 2-HG has been shown to inhibit α-KG-dependent enzymes involved in epigenetic regulation, collagen synthesis, and cell signaling, thereby leading to a block in normal differentiation of progenitor cells and the subsequent development of cancer. Therefore, inhibition of mutated IDH1 in tumor cells and the concomitant decrease in 2-HG production is a therapeutic approach to the treatment of IDH1-mutated cancers.

IDH1 mutations reported in cancer can occur at amino acid position R132, such as R132H, R132C, R132S, R132G, and R132L mutations. There remains a need for methods of identifying patients to receive therapeutically effective amount of an IDH1 inhibitor compound that selectively inhibits production of 2-HG from cancer cells harboring a variety of R132 IDH1 mutations.

### SUMMARY

Olutasidenib is a potent, selective, orally bioavailable, small-molecule inhibitor of mutated IDH1 and is useful as an anticancer therapeutic in patients with acute myeloid leukemia (AML) or myelodysplastic syndrome (MDS) with certain mutations in the IDH1 gene (including R132X mutations). Methods of administering olutasidenib for the treatment of certain hematological cancers (e.g., AML and MDS) are disclosed herein.

The present disclosure provides methods for the treatment of AML or MDS comprising a step of administering to a subject diagnosed with a cancer harboring a R132X IDH-1 mutation, a therapeutically effective amount of a pharmaceutically acceptable form of olutasidenib. In some examples, the pharmaceutically acceptable form of olutasidenib is an oral dosage form (e.g., as provided in Example 1). In some embodiments, olutasidenib is administered to the patient as R132X mIDH-1 Selective Inhibitor Therapy consisting of oral administration of an oral dosage form of olutasidenib administered either as a single agent inhibitor of mIDH-1, or in combination with azacitidine or cytarabine. When olutasidenib is administered in such combination therapy, the subject can be receiving or have previously received treatment with azacitidine or cytarabine.

Olutasidenib can be administered at a dose of 150 mg twice per day throughout the course of treatment. Olutasidenib can be administered with food to improve bioavailability of olutasidenib. The Course of Treatment can be at least 15 consecutive days starting with the initial dose of olutasidenib and longer (e.g., up to 30 weeks, 15 days to 30 weeks, 15 days to 12 weeks, at least 12 weeks, 12-30 weeks, 15 days to 6 months and other intermediate or longer durations or intervals apparent based on the present disclosure).

A patient can be identified as having a R132X mutation in mIDH1 using a diagnostic method disclosed herein prior to the administration of olutasidenib to the patient. The R132X gene mutation can be determined prior to administration of olutasidenib to the patient. Olutasidenib can be administered to patients who have received prior anticancer therapy or other concomitant (non-anticancer) medications. In some examples, olutasidenib is administered to patient who has not received a prior mIDH-1 inhibitor therapy.

### BRIEF DESCRIPTION OF THE DRAWINGS

FIG. 1A and FIG. 1B are schematics of the dysregulation of epigenetic and gene expression profiles associated with IDH mutants.

FIG. 2A is a graph of blood plasma concentration of Compound 1 (i.e., olutasidenib) measured in a group of human patients treated with Compound 1 as a single agent throughout a course of treatment. The dashed line labeled “C_{eff}” is lower than 1652 ng/mL.

FIG. 2B is a graph of blood plasma concentration of Compound 1 measured in a group of human patients treated with Compound 1 and azacitidine throughout a course of treatment. The dashed line labeled “C_{eff}” is lower than 1652 ng/mL.

FIG. 2C is a graph of effective blood plasma concentration of Compound 1 measured in a group of human patients throughout a course of treatment.

FIG. 2D is a graph of blood plasma concentration of Compound 1 measured in a group of human patients treated with Compound 1 and azacitidine throughout a course of treatment. The dashed line labeled “C_{eff}” is lower than 1652 ng/mL.

FIG. 2E is a graph of blood plasma concentration of Compound 1 measured in a group of human patients throughout a Course of Treatment.

FIG. 3 is a graph showing the levels of steady-state concentration of Compound 1 measured in a group of human patients at the day 15 trough (ng/mL) of Compound 1 after administration to patients of 150 mg QD, 300 mg QD, and 150 mg BID of Compound 1. FIG. 3 also illustrates steady state is achieved by Week 2; (t_{1/2}˜60 hrs) and 150 mg BID steady state exposures >IC₉₀ and below levels are expected to increase QTcF potential.

FIG. 4 is a graph Cₛₛ over time of patients treated with Compound 1 at 150 mg BID. FIG. 4 also illustrates that plasma exposures of Compound 1 are stable throughout treatment duration in patients. This can serve as basis for selecting a 150 mg BID dose for dose expansion and a recommended phase II dose.

FIG. 5A, FIG. 5B, and FIG. 5C are each a graph showing time on treatment of AML patients treated with a single agent (Compound 1).

FIG. 6A and FIG. 6B are each a graph showing time on treatment of AML patients treated with a combination of Compound 1 and azacitidine.

FIG. 7 is a synthetic reaction scheme for the preparation of Compound 1.

FIG. 8 depicts a dynamic vapor sorption (DVS) isotherm plot of Compound 1 Type A solid form.

FIG. 9 depicts a differential scanning calorimetry (DSC) thermogram for Compound 1 Type A solid form.

FIG. 10 depicts a thermogravimetric analysis (TGA) curve for Compound 1 Type A solid form.

FIG. 11 depicts X-ray powder diffraction (XRPD) of Compound 1 Type A solid form.

FIG. 12 consists of 4 panels: (a), (b), (c), and (d). Panel (a) illustrates free concentration of Compound 1 in plasma after three-dose oral administration (12.5, 25 and 50 mg/kg) with 12 hr dosing interval in mouse HCT116-IDH1-R132H/+ xenograft model. Panel (b) illustrates free concentration of Compound 1 in tumor after three-dose oral administration (12.5, 25 and 50 mg/kg) with 12 hr dosing interval in mouse HCT116-IDH1-R132H/+ xenograft model. Panel (c) illustrates percent 2-HG inhibition in tumors in a PO dose of 12.5 mpk, 25 mpk and 50 mpk at three different time points (4 h, 12 h, 24 h). Panel (d) illustrates in vivo activity (2-HG % inhibition) of Compound 1 vs free compound concentration in tumor.

FIG. 13 is a graph of the minimum blood plasma concentration (Cmin) of Compound 1 measured in groups of human patients receiving Compound 1 as a single agent at different dose amounts and dose intervals: day 15 trough (ng/mL) of Compound 1 after administration to patients of 100 QD, 150 mg QD, 300 mg QD, and 150 mg BID Compound 1 as a single agent. FIG. 13 also illustrates steady state is achieved by Week 2; (t_{1/2}˜60 hrs) and 150 mg BID and 300 mg QD steady state exposures are >C_{eff}.

FIG. 14 is a graph showing cycle 1, grade 2 IDH-DS is resolved with dexamethasone and hydroxyurea. C2D1: CRp (1% blasts) to SCT in MLFS.

FIG. 15A is a graph of % patients who achieved transfusion independence (28- and 56-day) by category (CR/CRh, Non-CR/CRh, non-responders and overall), illustrating transfusion independence by response category for patients treated with Compound 1 as a single agent.

FIG. 15B is a graph showing blasts maximum reduction from baseline (%) based on category (CR/CRh, CRi, SD and PD), illustrating marrow blast reduction for patients treated with Compound 1 as a single agent.

FIG. 16A is a graph of % patients who achieved transfusion independence (28- and 56-day) by category (CR/CRh, Non-CR/CRh, non-responders and overall), illustrating transfusion independence by response category for patients treated with Compound 1 in combination with azacitidine.

FIG. 16B is a graph showing blasts maximum reduction from baseline (%) based on category (CR/CRh, CRi, SD and PD), illustrating marrow blast reduction for patients treated with Compound 1 in combination with azacitidine.

FIG. 17 is a graph of the minimum blood plasma concentration (Cmin) of Compound 1 measured in groups of human patients receiving Compound 1 at different dose amounts and dose intervals, in combination with azacitidine: day 15 trough (ng/mL) of Compound 1 after administration to patients of 150 mg QD and 150 mg BID of Compound 1 in combination with azacitidine. FIG. 17 also illustrates steady state is achieved by Week 2; (t_{1/2}˜60 hrs) and 150 mg BID QD steady state exposures are >C_{eff}.

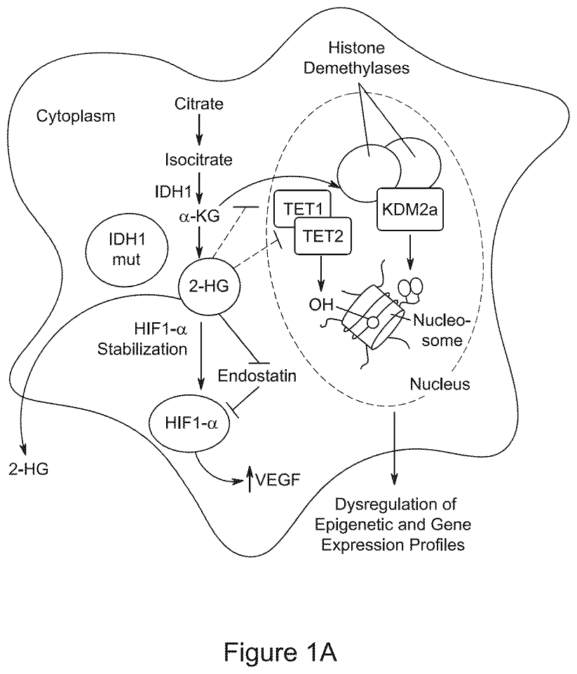

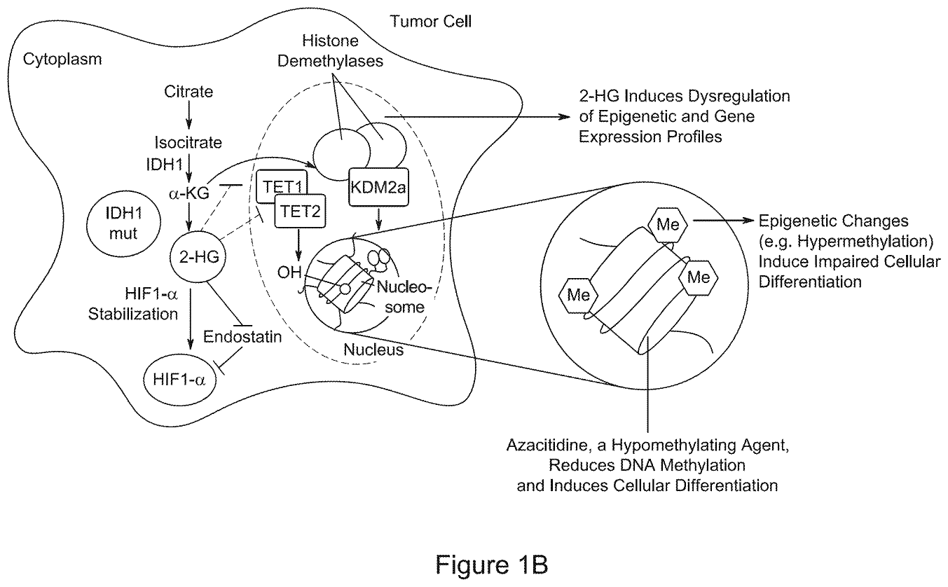

### DETAILED DESCRIPTION

### Definitions

As used herein, the term “R132X mIDH-1 mutation(s)” refers to a mutation at IDH-1 arginine 132 that results in inhibitory activity of Compound 1 against the mutated IDH-1 form harboring the R132 mutation. Preferably, the R132X mutations have a 2-HG IC₅₀ value of less than 500 nM (most preferably less than 250 nM or less than 150 nM) using the in vitro assay of Example 2. Accordingly, preferred R132X mutations include R132H and R132C, as well as R132L, R132G, and R132S (or other R132X mutations having therapeutically relevant 2-HG IC₅₀ values obtained using the in vitro assay of Example 2). Patients having R132X mIDH-1 mutation(s) can be identified using a suitable diagnostic, such as a diagnostic analyzing patient tissue with next generation sequencing technology that identified the presence of the R132X mIDH-1 mutation in the patient tissue sample.

As used herein, the term “R132X mIDH-1 Selective Inhibitor Therapy” refers to a therapy administered to a patient to inhibit the activity of R132X mIDH-1 in the patient, where the therapy is known to have selective inhibitory activity against R132X mIDH-1 over wild type IDH-1.

As used herein, the term “Course of Treatment” refers to the time period in which a patient is being administered an agent, including any administration holidays or recovery periods. A Course of Treatment can include a single treatment cycle or multiple treatment cycles. Additionally, a Course of Treatment can include a partial treatment cycle. The Course of Treatment can include the total time period during which a patient is on a treatment protocol for AML or MDS for a therapy comprising the administration of a mIDH-1 inhibitor compound.

“Next-generation sequencing” or “NGS” or “NG sequencing” as used herein, refers to any sequencing method that determines the nucleotide sequence of either individual nucleic acid molecules (e.g., in single molecule sequencing) or clonally expanded proxies for individual nucleic acid molecules in a high through-put fashion (e.g., greater than 103 or more molecules are sequenced simultaneously). Various next generation sequencing methods are known. In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. (2010) Nature Biotechnology Reviews 11:31-46, incorporated herein by reference. Next generation sequencing can detect a variant present in less than 5% of the nucleic acids in a sample.

The terms “subject” and “patient” are used interchangeably in the present disclosure.

### Provided Methods

In some embodiments, the present disclosure provides methods of treating acute myeloid leukemia (AML) in patients with an isocitrate dehydrogenase-1 (IDH1) mutation, the method comprising steps of:
a. providing DNA from a sample obtained from a patient;
b. detecting an IDH1 mutation in the DNA from the sample; and
c. administering to the patient with the IDH1 mutation a total of 150 mg of olutasidenib twice daily in a pharmaceutically acceptable composition.

In some embodiments, the present disclosure provides methods of treating acute myeloid leukemia (AML) in patients with an isocitrate dehydrogenase-1 (IDH1) mutation, the method comprising steps of:
a. isolating and purifying DNA from a sample obtained from a patient;
b. detecting an IDH1 mutation in the DNA from the sample; and
c. administering to the patient with the IDH1 mutation a total of 150 mg of olutasidenib twice daily in a pharmaceutically acceptable composition.

In some embodiments, the present disclosure provides methods of treating AML in patients with an isocitrate dehydrogenase-1 (IDH1) mutation, the method comprising steps of:
determining whether the patient has an IDH1 mutation by:
   i. obtaining a sample from the patient; and
   ii. performing an assay (e.g., an FDA-approved diagnostic test, such as the IDH1 Assay of Example 8) on the sample to determine if the patient has an IDH1 mutation; and
if the patient has an IDH1 mutation, then administering to the patient with the IDH1 mutation a total of 150 mg of olutasidenib twice daily in a pharmaceutically acceptable composition, and
if the patient does not have an IDH1 mutation, then not administering to the patient with the IDH1 mutation a total of 150 mg of olutasidenib twice daily in a pharmaceutically acceptable composition.

The present disclosure also provides methods of treating acute myeloid leukemia (AML) in patients with an isocitrate dehydrogenase-1 (IDH1) mutation, the method comprising administering twice daily to a patient with an IDH1 mutation 150 mg of olutasidenib in a pharmaceutically acceptable composition, wherein the IDH1 mutation has been detected using an FDA-approved diagnostic test.

In some embodiments, the IDH1 mutation is an IDH1 R132 mutation. Examples of an IDH1 R132 mutation include R132C, R132H, R132S, R132G, and R132L. In some embodiments, the IDH1 R132 mutation is R132C. In some embodiments, the IDH1 R132 mutation is R132H. In some embodiments, the IDH1 R132 mutation is R132S. In some embodiments, the IDH1 R132 mutation is R132G. In some embodiments, the IDH1 R132 mutation is R132L. In some embodiments, the patient is harboring an IDH1 mutation, such as an IDH1 R132 mutation selected from the group consisting of R132C, R132H, R132S, R132G, and R132L.

In some embodiments, the AML is relapsed or refractory AML.

In some embodiments, the patient is receiving or has received anticancer therapy. In some embodiments, the patient is resistant or refractory to prior anticancer therapy. In some embodiments, the patient is receiving or has received therapy comprising azacitidine or cytarabine.

Olutasidenib can be administered as provided herein. For example, olutasidenib can be administered as an oral dosage form, such as a tablet or a capsule. For example, olutasidenib can be administered as part of a combination therapy comprising, e.g., olutasidenib and azacitidine or cytarabine. For example, olutasidenib can be administered as the PRODUCT described in Example 7.

In some embodiments, provided methods further comprise not administering olutasidenib if the patient does not have an IDH1 mutation, as determined, e.g., by an FDA-approved diagnostic test.

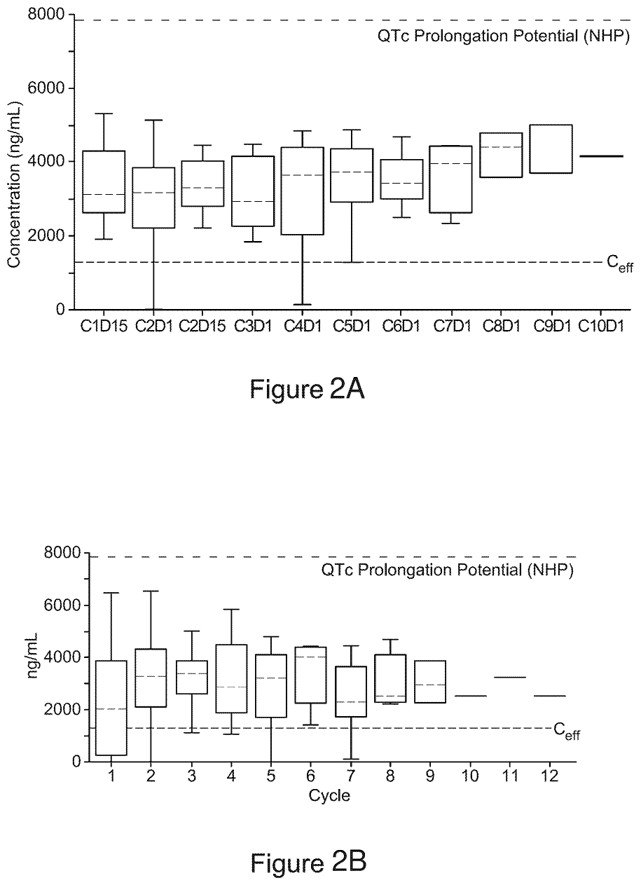

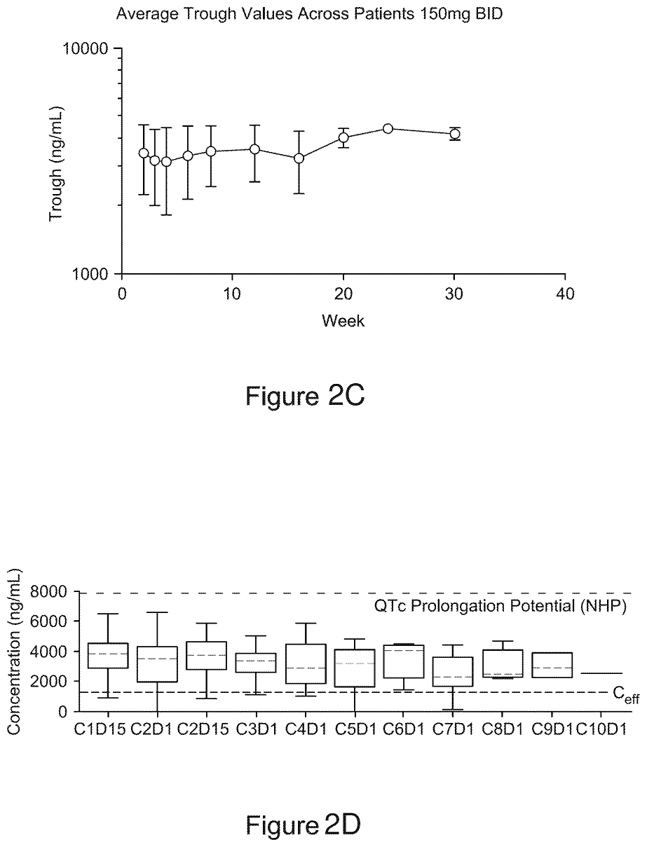

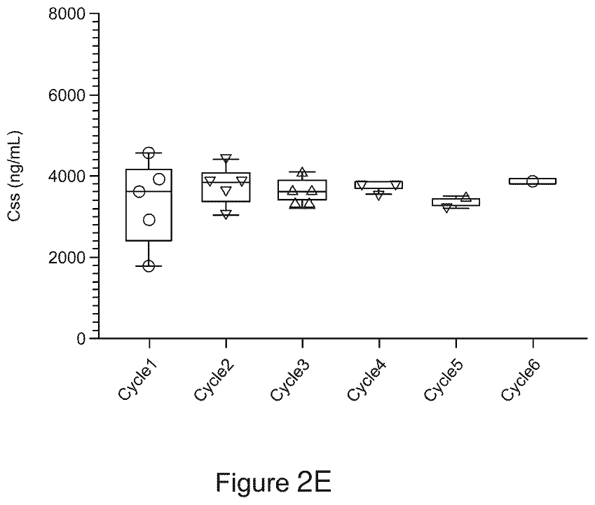

Administration of olutasidenib in the oral dosage form described in Example 1 at 150 mg BID resulted in a sustained therapeutically effective trough blood plasma concentration above 2,000 ng/mL after cycle 3 of a 28-day treatment cycle (FIGS. 2A-2E). The steady state blood concentrations of olutasidenib measured in the patients was above the IC₉₀ value for 2-HG inhibition in R132X mIDH-1 cells (described in the Examples). As shown in FIGS. 2A-2E, the plasma exposures (steady state blood plasma concentration) of olutasidenib were durable (i.e., sustained) throughout the 30-week treatment duration. No dose limiting toxicities of olutasidenib were observed during dose escalation studies, and the maximum tolerated dose (MTD) of olutasidenib was not reached.

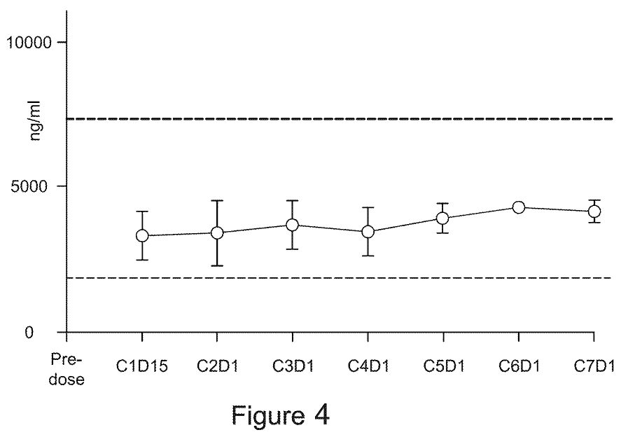

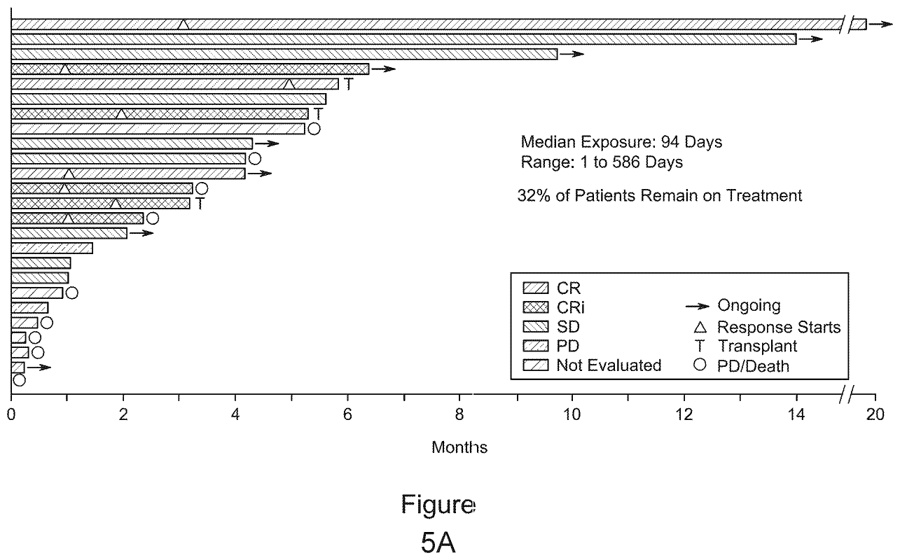

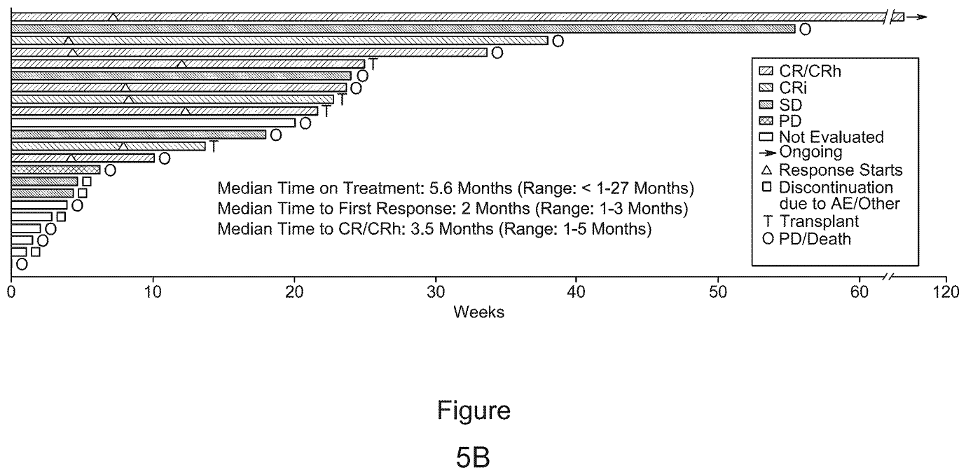

FIGS. 2A-2E are graphs of the data obtained from measuring the steady state concentrations from patients in the clinical trial receiving a 150 mg BID dose of olutasidenib, either as a single agent or in combination with azacitidine as described in Example 6. FIG. 4 is a graph showing the steady state concentration of olutasidenib measured in patients at various points during the Course of Treatment described in the clinical trial of Example 6, with each point representing a cycle number and day number (each cycle is 28 consecutive days of administration of 150 mg BID olutasidenib). The steady state concentration of olutasidenib remained above the minimum desired level (bottom dashed line) and the maximum desired level (upper dashed line).

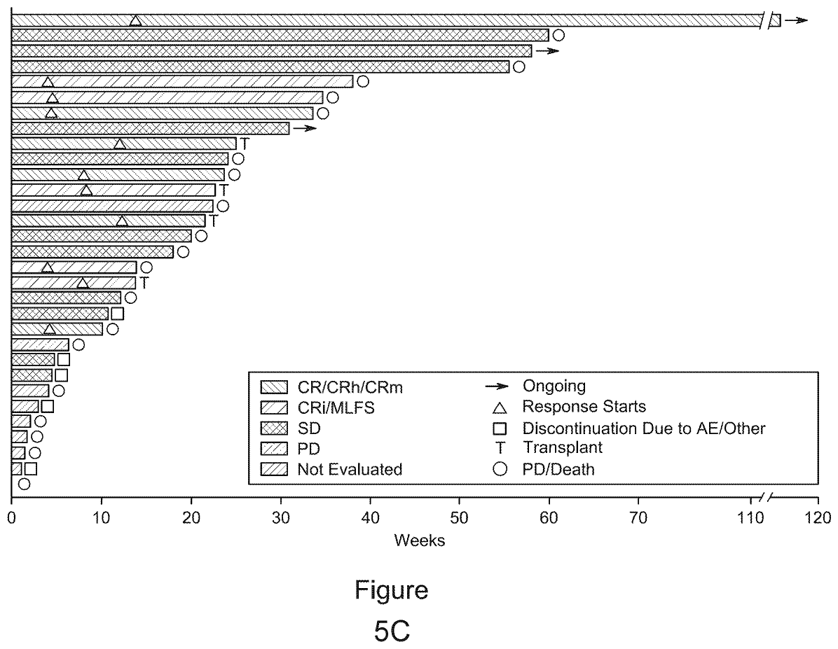

FIGS. 5A and 5C are graphs showing the results from the clinical trial in Example 6, showing the 150 mg BID administration of olutasidenib (solid form obtainable from Example 1) was administered as a single agent to multiple patients over a Course of Treatment times having a median of 94 days, and a range of 1 to 586 days. About 32% of the patients remain on treatment.

FIG. 5B depicts responses for relapsed or refractory (R/R) AML patients, and shows prolonged duration of treatment with olutasidenib was observed with a 1st response occurring within 2 months of treatment with olutasidenib. Responses <CR/CRi were noted to deepen with continued treatment resulting in CR/CRh/CRi rate of 41% in R/R AML. Clinical benefit (SD ≥8 weeks) was observed in subjects without an IWG defined response. 10% of patients (1 AML and 2 MDS) remained on treatment. Treatment Discontinuation: Progressive Disease (PD) (9), death (6), transplant (4), Adverse Events (AE) (3), investigator's decision (2), withdrawal of consent (1), and lack of response (3).

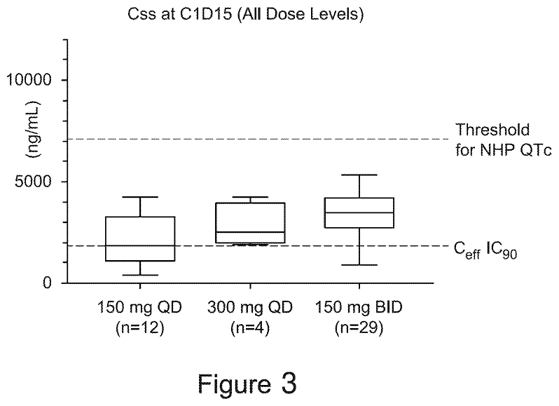

The invention is based in part on the discovery that administration of olutasidenib at 150 mg BID resulted in a higher blood exposure level than either 150 mg QD or 300 mg BID at day 15. See, for example, FIG. 3. Administration of olutasidenib at 150 mg QD BID resulted in a blood exposure level of <3000 ng/mL at day 15. Administration of olutasidenib at 300 mg QD BID did not result in improved blood levels at day 15. In contrast, administration of olutasidenib at 150 mg BID results in a blood exposure level of >3000 ng/mL at day 15.

The oral dosage form of olutasidenib (Example 1) was administered to human patients as a single agent (150 mg QD, 300 mg QD, 150 mg BID and 100 mg QD until disease progression) in a human clinical trial treating AML/MDS in cancer patients harboring a mIDH1 mutation, as described in the Examples below. FIG. 3 is a graph showing the concentration of olutasidenib measured in the blood of patients after receiving olutasidenib (as the solid form obtained from Example 1) in one of three different dose and dose intervals: 150 mg QD, 300 mg QD or 150 mg BID (either receiving olutasidenib as a single agent or in combination with azacitidine as described in the clinical trial of Example 6, in each category).

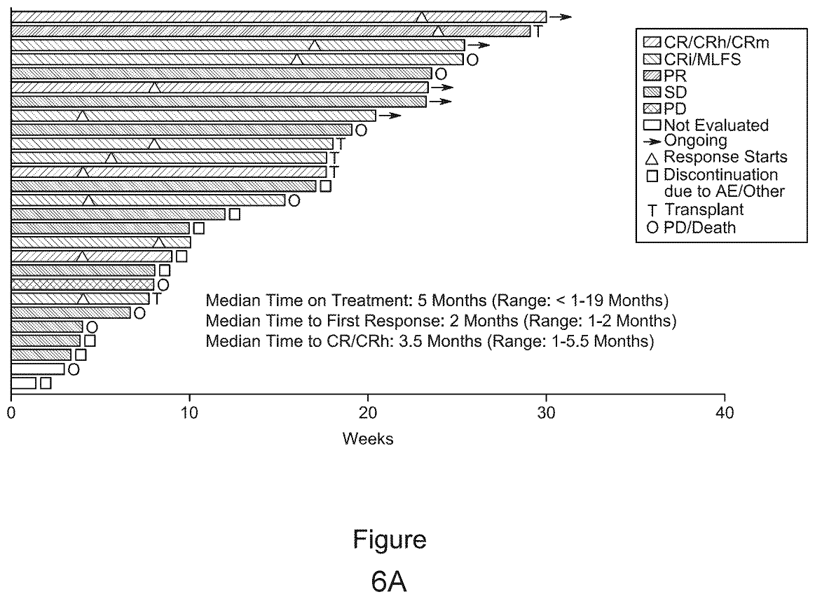

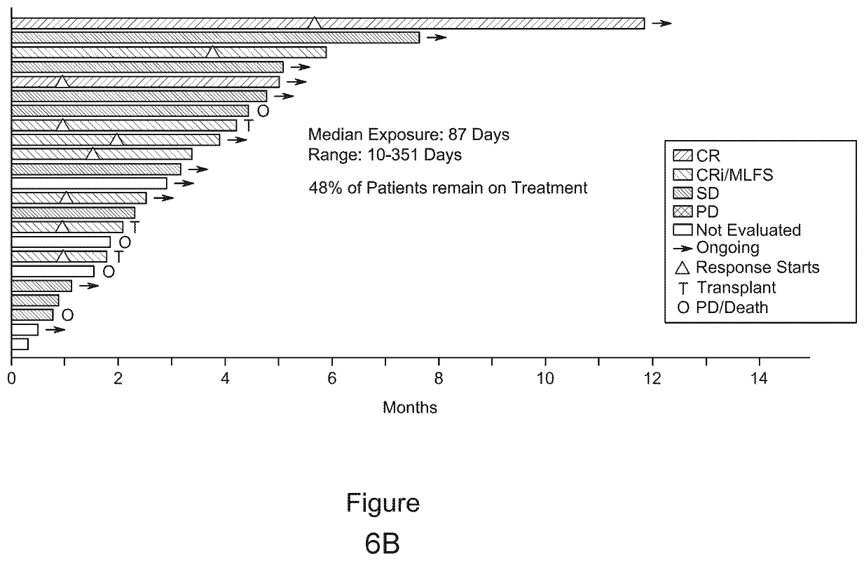

Olutasidenib can be administered to certain patients in combination with a hypomethylating agent such as azacitidine. IDH1 mutations (e.g., in AML or MDS patients harboring a R132X mIDH-1 mutation) can result in abnormal hypermethylation of histones and DNA and suppression of normal cellular differentiation. The combination of olutasidenib and azacitidine can be administered for the treatment of patients with AML harboring IDH1 mutations. For example, patients can be administered the olutasidenib daily (BID) in continuous 28-day cycles, alone or in combination with azacitidine (administered at the dose of 75 mg/m² for 7 days IV/SC per every 28-day cycle). For example, olutasidenib can be administered at a dose of 150 mg QD or 150 mg BID in combination with azacitidine (azacitidine administered per standard of care for a patient). FIG. 6B is a graph showing the results from the clinical trial in Example 6, showing the 150 mg BID administration of olutasidenib (solid form obtainable from Example 1) in combination with the administration of azacitidine (administered at the dose of 75 mg/m² for 7 days IV/SC per every 28-day cycle) were both administered to multiple patients over a Course of Treatment times having a median of 87 days, and a range of 10 to 351 days. About 48% of the patients remain on treatment.

As shown in FIG. 14A, upon further duration of treatment, clinical responses observed in R/R or TN AML and MDS: cytopenias associated with azacitidine may influence depth of IWG response. An ORR of 46% was observed for the combination of olutasidenib with azacitidine in R/R AML and an ORR of 78% was observed in TN AML (CR/CRi of 66%). Treatment Discontinuation was caused by: PD (6), transplant (6), investigator's decision (5), death (4), AE (2), and others: treatment failure, hospice, other treatment (1 each). 37% of patients (AML and MDS) remain on treatment.

In some methods, olutasidenib can be administered with cytarabine. Low dose cytarabine (LDAC) can be administered to certain AML patients (e.g., AML patients at or above about 60 years who are not candidates for intensive therapy, and harboring a R132X mIDH-1 mutation). The therapeutically effective combination of olutasidenib with LDAC can be administered to AML patients harboring IDH1 mutation. For example, patients can be administered olutasidenib daily (BID) in continuous 28-day cycles, alone or in combination with LDAC (administered at the dose of 20 mg BID SC for 10 days every 28-day cycle) until treatment discontinuation.

In some embodiments, the present disclosure additionally provides methods of treating AML or MDS in a patient harboring isocitrate dehydrogenase 1 mutations (mIDH1), which can comprise administering to a patient in need thereof a therapeutically effective amount of olutasidenib each day for at least three consecutive treatment cycles of 28 consecutive days each. The administration of the therapeutically effective amount of olutasidenib can result in the patient having a durable therapeutically effective trough blood plasma concentration of olutasidenib in the patient throughout the course of treatment.

### Diagnostic Tests

In some embodiments, provided methods comprise detecting an IDH1 mutation and administering olutasidenib as described herein. In some embodiments, IDH1 mutations can be detected using an FDA-approved diagnostic test, such as the IDH1 Assay described in Example 8.

In some embodiments, detecting an IDH1 mutation comprises detecting a single nucleotide variant (SNV) coding the IDH1 mutation. In some embodiments, the IDH1 mutation is selected from the group consisting of R132C, R132H, R132G, R132S, and R132L. In some embodiments, detecting an IDH1 R132C mutation comprises detecting the SNV: TGT. In some embodiments, detecting an IDH1 R132H mutation comprises detecting the SNV: CAT. In some embodiments, detecting an IDH1 R132G mutation comprises detecting the SNV: GGT. In some embodiments, detecting an IDH1 R132S mutation comprises detecting the SNV: AGT. In some embodiments, detecting an IDH1 R132L mutation comprises detecting the SNV: CTT.

In some embodiments, the IDH1 mutation is detected using PCR technology with homogenous real-time fluorescence detection. In some embodiments, the IDH1 mutation is detected using an in vitro polymerase chain reaction (PCR) assay for the qualitative detection of single nucleotide variants (SNVs) coding an IDH1 R132 mutation selected from the group consisting of R132C, R132H, R132G, R132S, and R132L in the DNA from a sample.

In some embodiments, the diagnostic test uses a sample obtained from the patient. In some embodiments, the sample is a blood or tissue sample. In some embodiments, the sample is patient bone marrow. In some embodiments, the sample is patient blood. In some embodiments, the sample has been preserved with EDTA.

In some embodiments, provided methods further comprise:
lysing cells from the sample at an elevated temperature in a lysis buffer comprising guanidine isothiocyanate;
capturing DNA released from the lysed cells using magnetic microparticles;
washing the captured DNA; and
eluting the captured DNA from the magnetic microparticles with elution buffer to give an extracted DNA sample.

In some embodiments, provided methods further comprise:
combining the extracted DNA sample, a DNA polymerase, oligonucleotide primers, deoxyribonucleoside triphosphates (dNTPs), and magnesium chloride in a well of a 96-well plate to give a mixture;
sealing the 96-well plate with a cover;
activating the DNA polymerase at a high temperature;
subjecting the mixture to thermal cycling comprising multiple rounds of heating (e.g., to a high temperature suitable to melt double-stranded DNA) and cooling (e.g., to a low temperature suitable to promote annealing of primers to their respective targets); and
measuring the real-time fluorescence signals of the mixture.

In some embodiments, the oligonucleotide primers are designed to specifically amplify (i) R132C and R132H mutations or (ii) R132G, R132S, and R132L mutations. In some embodiments, the oligonucleotide primers are designed to specifically amplify R132C and R132H mutations. In some embodiments, the oligonucleotide primers are designed to specifically amplify R132G, R132S, and R132L mutations. In some embodiments, two samples from the same patient are evaluated, so that one sample is mixed with oligonucleotide primers that are designed to specifically amplify R132C and R132H mutations and the other sample is mixed with oligonucleotide primers that are designed to specifically amplify R132G, R132S, and R132L mutations.

In some embodiments, the DNA polymerase is a thermophilic enzyme that has been chemically modified to render it inactive at ambient temperature.

In some embodiments, provided methods comprise an internal control. For example, in some embodiments, the mixture further comprises oligonucleotide primers designed to amplify a region of the IDH1 gene outside of codon 132, thereby serving as an endogenous internal control.

In some embodiments, the real-time fluorescence signal of each IDH1 mutation of either (i) R132C and R132H or (ii) R132G, R132S, and R132L is distinguishable in a single well. In some embodiments, the real-time fluorescence signal of the internal control and each IDH1 mutation of either (i) R132C and R132H or (ii) R132G, R132S, and R132L is distinguishable in a single well.

Without wishing to be bound by any particular theory, it may be desirable for a diagnostic test described herein to be performed in such a way as to prevent nucleic acid contamination. In some embodiments, the diagnostic test is performed in a sealed 96-well plate. In some embodiments, the diagnostic test is performed without opening the sealed 96-well plate. In some embodiments, aerosol barrier pipette tips are used for all pipetting in provided methods. In some embodiments, the diagnostic test is performed in a dedicated area.

The present disclosure enables one of skill in the relevant art to make and use the inventions provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description are by way of example to illustrate the discoveries provided herein.

### EXAMPLES

### Analytical Methods, Materials, and Instrumentation

Unless otherwise noted, reagents and solvents were used as received from commercial suppliers. Proton nuclear magnetic resonance (NMR) spectra were obtained on either Bruker or Varian spectrometers at 300 MHz. Spectra are given in ppm (δ) and coupling constants, J, are reported in Hertz. Tetramethylsilane (TMS) was used as an internal standard. Mass spectra were collected using a Waters ZQ Single Quad Mass Spectrometer (ion trap electrospray ionization (ESI)). High performance liquid chromatograph (HPLC) analyses were obtained using a XBridge Phenyl or C18 column (5 μm, 50×4.6 mm, 150×4.6 mm or 250×4.6 mm) with UV detection (Waters 996 PDA) at 254 nm or 223 nm using a standard solvent gradient program (Methods 1-2).

### LCMS Method 1 (ESI. 4 Min Method):

| Instruments: | |
|---|---|
| HPLC: Waters HT2790 Alliance | MS: Waters ZQ Single Quad Mass Spectrometer |
| UV: Waters 996 PDA | |
| Conditions: | |
| Mobile phase A | 95% water/5% methanol with 0.1% Formic Acid |
| Mobile phase B (B) | 95% methanol/5% water with 0.1% Formic Acid |
| Column | XBridge Phenyl or C18, 5 pm 4.6 x 50 mm |
| Column temperature | Ambient |
| LC gradient | Linear 5-95% B in 2.5 min, hold 95% B to 3.5 min |
| LC Flow rate | 3 mL/min |
| UV wavelength | 220 nm and 254 nm |
| Ionization Mode | Electrospray Ionization; positive/negative |

### LCMS Method 2: (APCI, 20 Min):

### Instruments and Conditions:

HPLC-Agilent 1100 series.
Column: Agela Technologies Durashell C18, 3 μm, 4.6×50 mm,).
Mobile Phase A: acetonitrile+0.1% trifluoroacetic acid.
Mobile Phase B: Water+0.1% trifluoroacetic acid.

| Gradient: | |
|---|---|
| Time (min) | % B |
| 00 | 95 |
| 15 | 05 |
| 18 | 05 |
| 20 | 95 |

Flow Rate: 1 ml/min.
Column Temperature: Ambient.
Detector: 254 nm.

### Example 1—Synthesis of (S)-5-((1-(6-chloro-2-oxo-1,2-dihydroquinolin-3-yl)ethyl)amino)-1-methyl-6-oxo-1,6-dihydropyridine-2-carbonitrile (Compound 1)

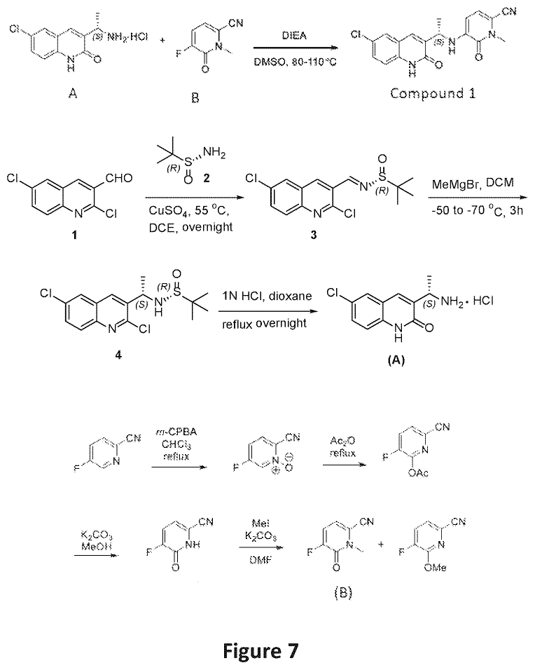

Compound 1 can be prepared in a convergent synthesis from Intermediate A and Intermediate B as shown in FIG. 7 via a nucleophilic displacement reaction under basic conditions of (S)-3-(1-aminoethyl)-6-chloroquinolin-2(1H)-one (Intermediate A) and the fluoropyridone (Intermediate B). ¹H, ¹³C NMR and mass spectral data are consistent with the assigned structure. Compound 1 (5-{[(1S)-1-(6-chloro-2-oxo-1,2-dihydroquinolin-3-yl)ethyl]amino}-1-methyl-6-oxo-1,6-dihydropyridine-2-carbonitrile) has a molecular weight of 355 with a melting point onset temperature of 251.3° C. (DSC) and peak maximum 254.1° C.

The asymmetric synthesis of Intermediate A started with the condensation of the commercially available quinoline aldehyde (1) with (R)-tert-butanesulfinamide (2) to form the chiral (R)-N-tert-butanesulfinimine (3), followed by addition of methyl magnesium bromide in dichloromethane to yield the desired product (4) as the major diastereoisomer (dr: 98:2). Cleavage of the chiral auxiliary and simultaneous hydrolysis of 2-chloroquinoline moiety under mildly acidic conditions using 1N HCl in dioxane gave Intermediate A in quantitative yield. The structure of Intermediate A was confirmed by NMR and mass spectroscopy, and the enantiomeric purity was determined by chiral SFC analysis. The (5)-stereochemistry was confirmed by X-ray co-crystal structures of several inhibitor analogs prepared from the same chiral amine intermediate bound to mIDH-1 R132H.

Intermediate (B) was prepared from commercially available 5-fluoropicolinonitrile in four steps. N-oxidation of 5-fluoropicolinonitrile followed by reflux of the N-oxide in acetic anhydride gave the acetate, following work-up and purification. Solvolysis of the acetate group followed by N-methylation under standard conditions gave a mixture of N-methylated and O-methylated products (4:1). The minor O-methylated product was removed by column chromatography. NMR and mass spectral data are consistent with the structure of Intermediate Compound (B).

### Intermediate 1: (S)-3-(1-aminoethyl)-6-chloroquinolin-2(1H)-one hydrochloride (A)

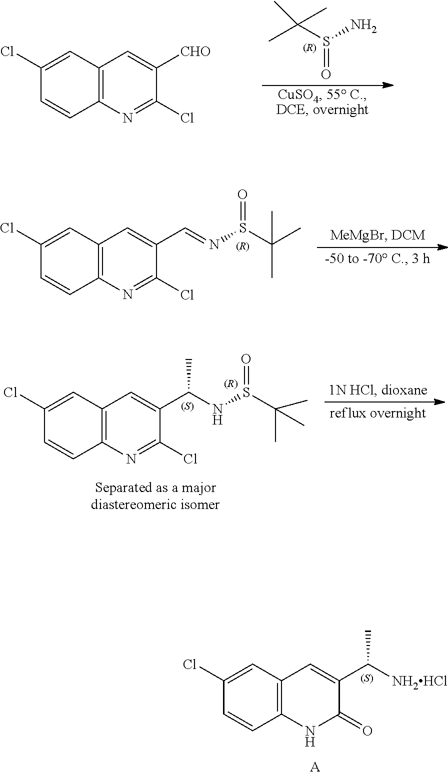

### Step-1: (R,E)-N-((2,6-dichloroquinolin-3-yl) methylene)-2-methylpropane-2-sulfinamide

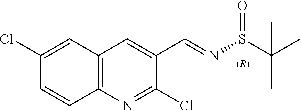

To a mixture of 2,6-dichloroquinoline-3-carbaldehyde (15.0 g, 66.37 mmol) and (R)-2-methylpropane-2-sulfinamide (8.85 g, 73.14 mmol) in 1,2-dichloroethane (150 mL) was added CuSO₄ (16.0 g, 100.25 mmol). The resulting mixture was heated to 55° C. and stirred at 55° C. overnight. After TLC and MS showed complete disappearance of starting materials, the mixture was cooled to room temperature and filtered through a pad of Celite®. The pad of Celite® was then rinsed with CH₂Cl₂. The filtrate was evaporated to dryness in vacuo and purified by SiO₂ column chromatography (0 to 25% hexanes/EtOAc) to afford the title compound, (R,E)-N-((2,6-dichloroquinolin-3-yl)methylene)-2-methylpropane-2-sulfinamide, as a yellow solid (17.7 g, 81% yield).

### Step-2: (R)-N-((S)-1-(2,6-dichloroquinolin-3-yl)ethyl)-2-methylpropane-2-sulfinamide

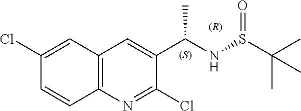

To a solution of (R,E)-N-((2,6-dichloroquinolin-3-yl)methylene)-2-methylpropane-2-sulfinamide (8.85 g, 26.88 mmol) in anhydrous CH₂Cl₂ (200 mL) at −60° C. was added dropwise MeMgBr (3M solution in diethyl ether, 13.5 mL, 40.54 mmol). The resulting reaction mixture was stirred at about −60 to −50° C. for 3 hours and then stirred at −20° C. overnight under an atmosphere of N₂. After TLC and MS showed complete disappearance of starting materials, saturated NH₄Cl (163 mL) was added at −20° C. and the resulting mixture was stirred for 10 minutes. The aqueous phase was extracted with CH₂Cl₂ (100 mL×3), dried over anhydrous Na₂SO₄, filtered, and evaporated. The residue was purified by column chromatography on an ISCO® chromatography system (SiO₂: Gold column; gradient; hexanes to 100% EtOAc) to provide the title compound, (R)-N-((S)-1-(2,6-dichloroquinolin-3-yl)ethyl)-2-methylpropane-2-sulfinamide, as a yellow solid (5.8 g, 63% yield).

### Step-3: (S)-3-(1-aminoethyl)-6-chloroquinolin-2(1H)-one hydrochloride (A)

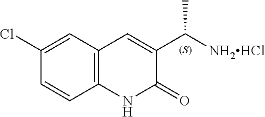

A mixture of (R)-N-((S)-1-(2,6-dichloroquinolin-3-yl)ethyl)-2-methylpropane-2-sulfinamide (6.6 g, 19.13 mmol) in 1,4-dioxane (41 mL) and 1N HCl (41 mL) was heated at reflux overnight. The solvents were evaporated in vacuo and the resulting residue was dissolved in hot water and lyophilized. The crude product was triturated with diethyl ether to afford the title compound A as a yellow solid (9.0 g, ee: 98.4%). ¹H NMR (300 MHz, DMSO-d₆): δ ppm 12.4 (br s, 1H), 8.32 (br s, 2H), 8.07 (s, 1H), 7.85 (d, J=2.2 Hz, 1H), 7.63 (dd, J1=8.8 Hz, J2=2.5 Hz, 1H), 7.40 (d, J=8.8 Hz, 1H), 4.40-4.45 (m, 1H), 1.53 (d, J=8.5 Hz, 3H). LCMS (Method 2): Rt 3.42 min, m/z 223.1 [M+H]⁺.

### Intermediate 2: 5-fluoro-1-methyl-6-oxo-1,6-dihydropyridine-2-carbonitrile (B)

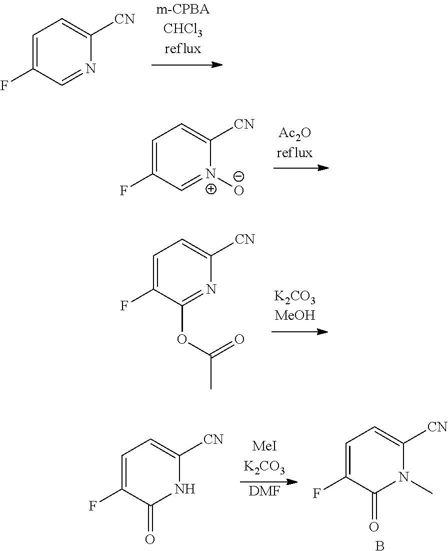

### Step-1: 2-cyano-5-fluoropyridine 1-oxide

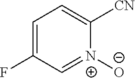

A solution of 5-fluoropicolinonitrile (7.27 g, 59.5 mmol) in CHCl₃ (60 mL) was added dropwise by addition funnel to a solution of m-CPBA (<77%, 22.00 g, 98 mmol) in CHCl₃ 160 mL). The solution was stirred at reflux for 4 days, at which time LCMS showed ˜85% conversion. The sample was allowed to cool, then sodium sulfite (12.4 g, 98 mmol) was added and the sample was stirred at room temperature for three hours, during which time the solution became thick with a white precipitate. The sample was diluted with DCM (300 mL) and filtered on a Buchner funnel, and the filter cake was washed with DCM (˜400 mL). A white material precipitated in the filtrate. The filtrate mixture was washed with saturated aqueous NaHCO₃ (400 mL), during which the solids went into solution. The organic layer was washed with water (300 mL), then dried (MgSO₄) and filtered. Silica gel was added and the mixture was evaporated under reduced pressure. The material was chromatographed by Biotage MPLC (340 g silica gel column) with 0 to 100% EtOAc in hexanes, with isocratic elution when peaks came off to provide 2-cyano-5-fluoropyridine 1-oxide (4.28 g, 31.0 mmol, 52% yield) as a white solid. ¹H NMR (300 MHz, DMSO-d₆): δ ppm 8.85-8.93 (m, 1H), 8.23 (dd, J=9.09, 6.74 Hz, 1H), 7.53-7.64 (m, 1H). LCMS (Method 1): Rt 0.57 min., m/z 138.9 [M+H]⁺.

### Step 2: 6-cyano-3-fluoropyridin-2-yl acetate

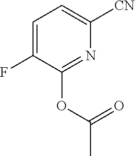

A solution of 2-cyano-5-fluoropyridine 1-oxide (4.28 g, 31.0 mmol) in acetic anhydride (40 ml, 424 mmol) was heated at reflux (150° C. bath) three days, during which the clear solution turned dark. The sample was concentrated under reduced pressure. The residue was dissolved in MeOH (30 mL) and stirred 1 hour. Silica gel was added and the solvent was evaporated under reduced pressure. The material was chromatographed by Biotage MPLC (100 g silica gel column) with 0 to 23% EtOAc in hexanes to provide 6-cyano-3-fluoropyridin-2-yl acetate (3.32 g, 18.43 mmol, 60% yield) as a clear liquid that solidified on cooling. 1H NMR (300 MHz, CHLOROFORM-d): δ ppm 7.65-7.75 (m, 2H), 2.42 (s, 3H). LCMS (Method 1): Rt 1.54 min., m/z 138.8 (loss of acetate).

### Step 3: 5-fluoro-6-oxo-1,6-dihydropyridine-2-carbonitrile

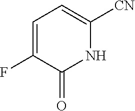

A solution of 6-cyano-3-fluoropyridin-2-yl acetate (3.32 g, 18.43 mmol) in MeOH (40 ml) was treated with potassium carbonate (5.10 g, 36.9 mmol) and stirred at room temperature for four hours. LCMS at 2 hours showed the reaction had gone to completion. The solvent was evaporated under reduced pressure. The residue was dissolved in water (100 mL) and acidified to pH ≤1 with 1M HCl. The solution was extracted with EtOAc (3×100 mL). The combined organic extracts were dried (Na₂SO₄), filtered, and evaporated under reduced pressure to provide 5-fluoro-6-oxo-1,6-dihydropyridine-2-carbonitrile (2.34 g, 16.94 mmol, 92% yield) as a white solid. ¹H NMR (300 MHz, DMSO-d₆): δ ppm 12.92 (br s, 1H), 7.73 (br s, 1H), 7.43 (br s, 1H). LCMS (Method 1): Rt 0.70 min., m/z 138.9 [M+H]⁺.

### Step 4: 5-fluoro-1-methyl-6-oxo-1,6-dihydropyridine-2-carbonitrile (B)

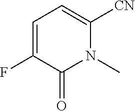

A mixture of 5-fluoro-6-oxo-1,6-dihydropyridine-2-carbonitrile (2.31 g, 16.73 mmol) and potassium carbonate (4.86 g, 35.2 mmol) in a 200 mL round bottom flask was treated with DMF (46 mL) and stirred 15 minutes. Mel (1.2 mL, 19.19 mmol) was added and the mixture was stirred at room temperature 45 minutes. The solvent was evaporated under reduced pressure. The residue was mixed with water (150 mL) and extracted with DCM (2×150 mL). The combined organic extracts were dried (MgSO₄), filtered, treated with silica gel, and evaporated under reduced pressure, then evaporated further at 60° C. under high vacuum. The material was chromatographed by Biotage MPLC with 0 to 35% EtOAc in hexanes, with isocratic elution at 16% EtOAc and 35% EtOAc while peaks came off. The peak that came off with 16% EtOAc was O-methylated material and was discarded. The peak that came off with 35% EtOAc provided the title compound B (1.70 g, 11.17 mmol, 67% yield) as a solid. ¹H NMR (300 MHz, DMSO-d₆): δ ppm 7.53 (dd, J=9.38, 7.62 Hz, 1H), 7.18 (dd, J=7.77, 4.84 Hz, 1H), 3.60 (s, 3H). LCMS (Method 1): Rt 0.94 min., m/z 152.9 [M+H]⁺.

### Step 5: (S)-5-((1-(6-chloro-2-oxo-1,2-dihydroquinolin-3-yl)ethyl)amino)-1-methyl-6-oxo-1,6-dihydropyridine-2-carbonitrile (Compound 1)

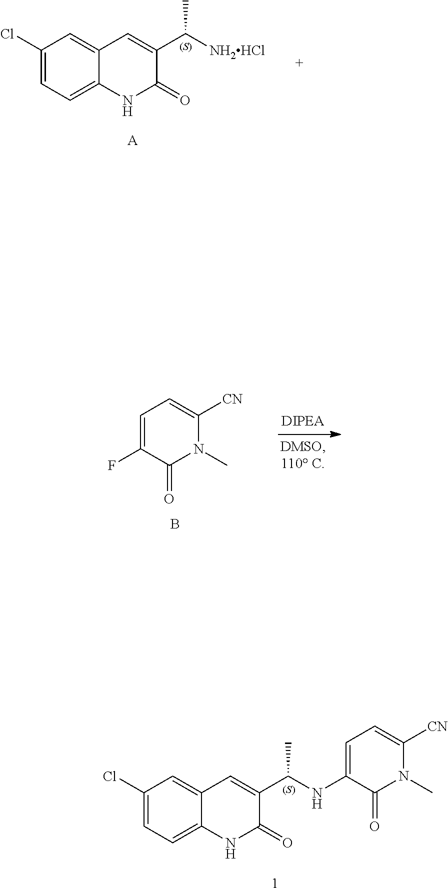

A mixture of 5-fluoro-1-methyl-6-oxo-1,6-dihydropyridine-2-carbonitrile B (1.23 g, 8.09 mmol), (S)-3-(1-aminoethyl)-6-chloroquinolin-2(1H)-one hydrochloride A (1.91 g, 7.37 mmol) and N,N-diisopropylethylamine (3.8 mL, 21.8 mmol) in anhydrous dimethyl sulfoxide (57 mL) under N₂ was heated to 110° C. and stirred for 6 hours. After cooling to room temperature, the mixture was partitioned between EtOAc/H₂O (750 mL/750 mL). The organic layer was separated, dried (Na₂SO₄) and concentrated in vacuum. The residue was purified on ISCO twice (40 g silica gel column, EtOAc/hexanes 0-100%; 80 g silica gel column, MeOH/dichloromethane 0-5%). The colorless fractions were combined and dichloromethane was removed under reduced pressure on rotavap until a lot of white solid precipitated out. The white solid was collected by filtration and washed with cold MeOH. It was then mixed with MeCN/H₂O (10 mL/25 mL) and lyophilized to afford the title Compound 1 as a white solid (790 mg). m.p. 262-264° C. ¹H NMR (300 MHz, DMSO-d₆) δ: 12.07 (s, 1H), 7.75 (s, 1H), 7.73 (d, J=2.2 Hz, 1H), 7.51 (dd, J=8.6, 2.3 Hz, 1H), 7.31 (d, J=8.8 Hz, 1H), 6.97 (d, J=8.0 Hz, 1H), 6.93 (d, J=7.7 Hz, 1H), 5.95 (d, J=8.0 Hz, 1H), 4.68 (m, 1H), 3.58 (s, 3H), 1.50 (d, J=6.6 Hz, 3H). LCMS (Method 2): 100% pure @254 nm, Rt 10.78 min, m/z 355, 357 [M+H]+. The filtrate and the colored fractions (TLC pure) from the second ISCO were combined and treated with activated charcoal and filtered (until the filtrate was colorless). The filtrate was then concentrated under reduced pressure on rotavap to remove dichloromethane until a lot of white solid precipitated out. The white solid was collected by filtration and washed with cold MeOH. It was then mixed with MeCN/H₂O (10 mL/25 mL) and lyophilized to afford the title Compound 1 as a white solid (970 mg). m.p. 262-264° C. ¹H NMR (300 MHz, DMSO-d₆) δ: 12.06 (s, 1H), 7.75 (s, 1H), 7.73 (d, J=2.5 Hz, 1H), 7.51 (dd, J=8.6, 2.3 Hz, 1H), 7.31 (d, J=8.8 Hz, 1H), 6.97 (d, J=8.0 Hz, 1H), 6.92 (d, J=8.0 Hz, 1H), 5.95 (d, J=8.0 Hz, 1H), 4.68 (m, 1H), 3.58 (s, 3H), 1.50 (d, J=6.9 Hz, 3H). LCMS (Method 2): 100% pure @254 nm, m/z 355, 357 [M+H]+. The total yield for combined two batches is 67%.

Compound 1 is also known as olutasidenib (see Example 7).

### Step 6: Solid form of (S)-5-((1-(6-chloro-2-oxo-1,2-dihydroquinolin-3-yl)ethyl)amino)-1-methyl-6-oxo-1,6-dihydropyridine-2-carbonitrile (Compound 1)

Unless otherwise indicated, the clinical trial in Example 6 was performed using a pharmaceutically acceptable solid form of Compound 1 in an oral dosage form that can be obtained by the method of Step 6 of Example 1. All volumes are with respect to the quantity of Compound 1 (v/w). Compound 1 is dissolved in 18 volumes of dichloromethane. The resulting solution is then concentrated under reduced pressure to approximately 5 volumes. To the mixture is added 5 volumes of ethyl acetate. The mixture is concentrated under reduced pressure to 5 volumes. To the mixture is added an additional 5 volumes of ethyl acetate, and the mixture again concentrated under reduced pressure to 5 volumes. The mixture is diluted to 10 volumes with ethyl acetate, and the mixture stirred at room temperature for 18 hours and then cooled to 0° C. The mixture is stirred at 0° C. for 3 hours and then filtered. The solids are rinsed with ethyl acetate and dried under vacuum (counterbalanced by nitrogen) at ambient temperature.

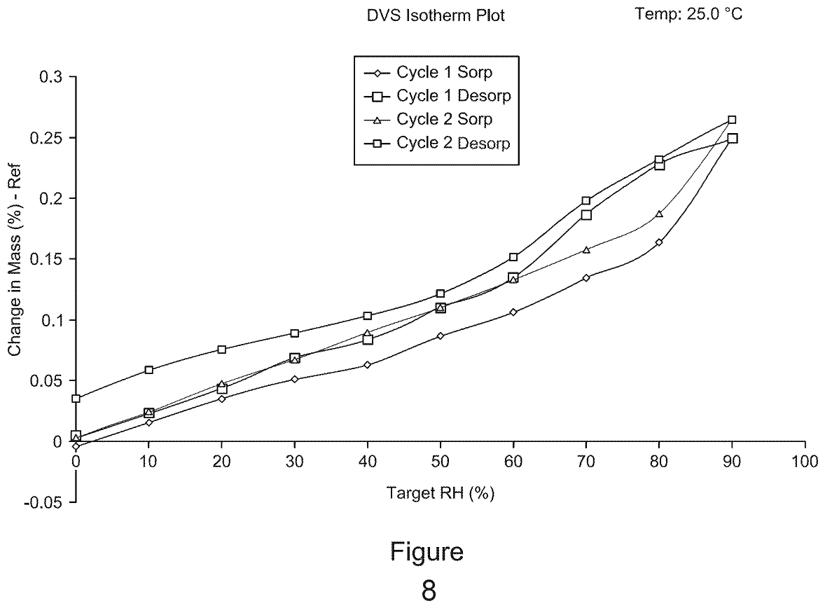

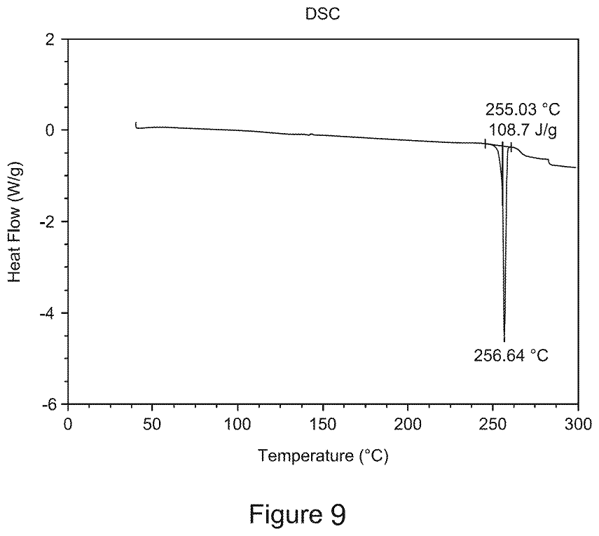

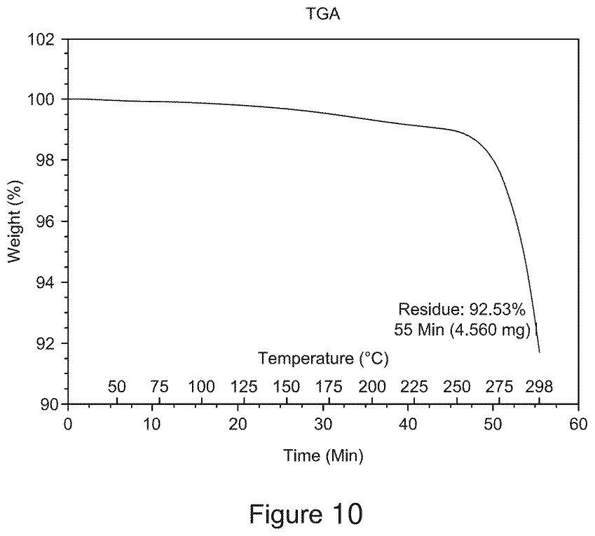

The crystalline solid was determined to be the solid form of Compound 1 Type A. The DVS Isotherm of Compound 1 Type A is shown in FIG. 8. DVS shows maximum water uptake of 0.25% w/w at 25° C./90% RH, indicating Compound 1 Type A is not hygroscopic. The thermal behavior Compound 1 Type A was evaluated using DSC. An endothermic event was observed at 256.6° C. (peak max). The onset temperature and heat of fusion were 255.0° C. and 108.7 J/g respectively (FIG. 9). TGA data (FIG. 10) do not show significant release of moisture or nonaqueous residual volatiles from Compound 1 Type A.

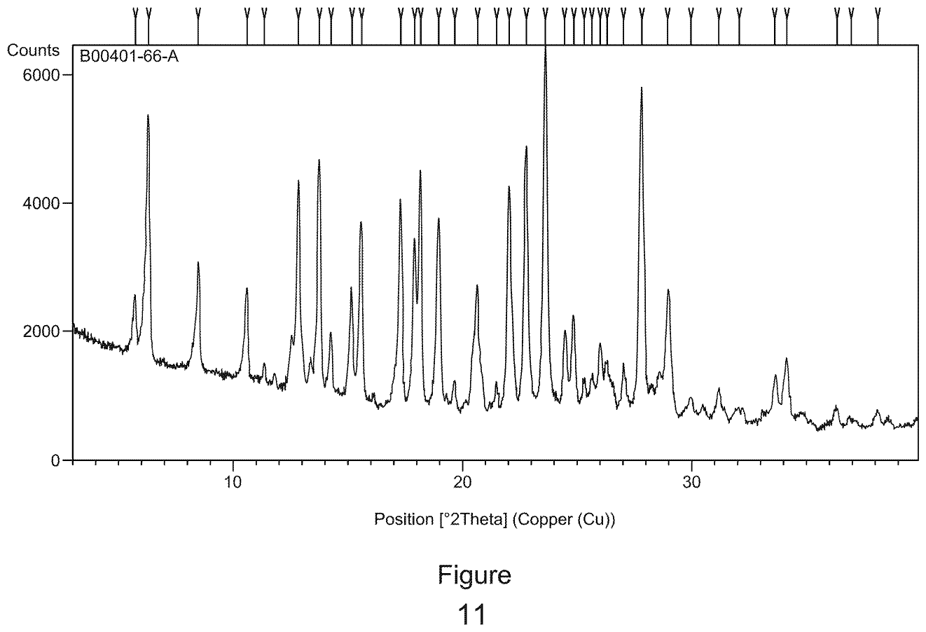

The X-ray powder diffraction pattern of the crystalline Compound 1 Type A is depicted in FIG. 11, and the corresponding data is summarized in Table 1 below.

TABLE 1

| 2 theta ± | d-spacing |
|---|---|
| 0.2 | Å ± 0.2 |
| 5.7 | 15.4 |
| 6.3 | 14.0 |
| 8.5 | 10.4 |
| 10.6 | 8.4 |
| 11.4 | 7.8 |
| 12.8 | 6.9 |
| 13.8 | 6.4 |
| 14.2 | 6.2 |
| 15.2 | 5.8 |
| 15.6 | 5.7 |
| 17.3 | 5.1 |
| 17.9 | 5.0 |
| 18.2 | 4.9 |
| 18.9 | 4.7 |
| 19.6 | 4.5 |
| 20.6 | 4.3 |
| 21.5 | 4.1 |
| 22.0 | 4.0 |
| 22.8 | 3.9 |
| 23.6 | 3.8 |
| 24.5 | 3.6 |
| 24.8 | 3.6 |
| 25.3 | 3.5 |
| 25.6 | 3.5 |
| 26.0 | 3.4 |
| 26.3 | 3.4 |
| 27.0 | 3.3 |
| 27.8 | 3.2 |
| 28.9 | 3.1 |
| 30.0 | 3.0 |
| 31.2 | 3.0 |
| 32.1 | 2.8 |
| 33.6 | 2.7 |
| 34.1 | 2.6 |
| 36.3 | 2.5 |
| 37.0 | 2.4 |
| 38.1 | 2.4 |

Preferably, the oral dosage form of Compound 1 is a solid form designated Type A that is characterized by a reflection X-ray powder diffraction (XRPD) pattern comprising characteristic peaks at 6.3, 12.8, 13.8, 23.6, and 27.8 degrees±0.2° 28. High resolution X-ray Powder Diffraction experiments were performed with Panalytical X'Pert3 Powder XRPD on a Si zero-background holder. The 2 theta position was calibrated against Panalytical 640 Si powder standard. Details of the XRPD method are listed below, with XRPD peaks reported as diffraction angles at 2 theta, with d-spacing measured in angstroms.

Parameters for Reflection Mode

| X-Ray wavelength | Cu, kα, Kα1 (Å): 1.540598, Kα2 (Å): 1.544426, |
|---|---|
| | Kα2/Kα1 intensity ratio: 0.50 |
| X-Ray tube setting | 45 kV, 40 mA |
| Divergence slit | Automatic |
| Scan mode | Continuous |
| Scan range (°2TH) | 3°-40° |
| Step size (°2TH) | 0.0131 |
| Scan speed (°/s) | 0.033 |

Compound 1 is preferably administered in an oral unit dosage form comprising a pharmaceutical composition that includes the following formulation: (a) Type A solid form of Compound 1 (e.g., in a relative weight of about 33), (b) a microcrystalline cellulose (e.g., in a relative weight of about 61), (c) a croscamellose sodium (e.g., in a relative weight of about 5) and a magnesium stearate (e.g., in a relative weight of about 1). The pharmaceutical composition for oral administration can comprise Compound 1 (e.g. in a Type A solid form) with pharmaceutically acceptable excipients in a capsule or tablet. For example, a capsule may contain a total of 50 mg or 150 mg of Compound 1 in a unit dosage form. The capsule may encapsulate the pharmaceutical composition comprising Compound 1 in a relative weight of about 30-50% by weight relative to the weight of the pharmaceutical composition. In another embodiment, a GMP manufacturing batch can comprise Compound 1, optionally provided in the Type A solid form.

In particular, the Compound 1 Type A solid form can be characterized by an X-ray Powder Diffraction (XRPD), having diffractions at angles (2 theta±0.2) of 6.3, 12.8, 13.8, 23.6, and 27.8. In some embodiments, a novel Compound 1 Type A is characterized by an X-ray Powder Diffraction (XRPD), having diffractions at angles (2 theta±0.2) of 6.3, 12.8, 13.8, 23.6, and 27.8, corresponding to d-spacing (angstroms±0.2) of 14.0, 6.9, 6.4, 3.8, and 3.2, respectively. In some embodiments, Compound 1 Type A can be identified by X-ray Powder Diffraction (XRPD), having characteristic diffractions at angles (2 theta±0.2) of 5.7, 6.3, 8.5, 10.6, 12.8, 13.8, 17.3, 22.0, 22.8, 23.6, and 27.8. In some embodiments, Compound 1 Type A can be identified by X-ray Powder Diffraction (XRPD), having characteristic diffractions at angles (2 theta±0.2) of 5.7, 6.3, 8.5, 10.6, 12.8, 13.8, 17.3, 22.0, 22.8, 23.6, and 27.8, corresponding to d-spacing (angstroms±0.2) of 15.4, 14.0, 8.4, 6.9, 6.4, 5.1, 4.0, 3.9, 3.8, and 3.2, respectively.

In some embodiments, Compound 1 Type A solid form is characterized by a differential scanning calorimetry (DSC) endotherm having a minima at about 256.64° C. Differential Scanning Calorimetry (DSC) experiments were performed on TA Q2000 DSC from TA Instruments. Samples were heated at 10° C./min from about 20° C. to about 300° C. using dry nitrogen to purge the system. The details of the method are provided below:

| Parameters | DSC |
|---|---|
| Pan Type | Aluminum pan, closed |
| Temperature | RT-250° C. |
| Ramp rate | 10° C./min |
| Purge gas | N₂ |

### Step 7: Oral solid dosage form of (S)-5-((1-(6-chloro-2-oxo-1,2-dihydroquinolin-3-yl)ethyl)amino)-1-methyl-6-oxo-1,6-dihydropyridine-2-carbonitrile

The oral dosage form of Compound 1 is a pharmaceutically acceptable solid form of the compound (S)-5-((1-(6-chloro-2-oxo-1,2-dihydroquinolin-3-yl)ethyl)amino)-1-methyl-6-oxo-1,6-dihydropyridine-2-carbonitrile, can be obtained using the method of Example 1 Step 6. The solid form of Compound 1 used in the oral dosage form does not contain associated solvent or a counter ion. In particular, the oral dosage form of Compound 1 can be a capsule comprising drug substance (Compound 1) blended with excipients to improve powder flow and encapsulated in a Coni-Snap® hard gelatin capsule suitable for oral dosage in humans.

### Example 2—In Vitro Activity of Compound 1 as a R132X mIDH-1 Inhibitor

In in vitro biochemical assays, Compound 1 significantly inhibited mutated IDH1-R132H and IDH1-R132C proteins. In contrast, Compound 1 displayed little or no inhibitory activity in biochemical assays of wild-type IDH1 protein or various mutated IDH2 proteins found in human cancers. Compound 1 suppressed 2-HG production in naturally occurring and genetically engineered cell lines expressing five different mutated IDH1 proteins (R132H, R132C, R132G, R132L, and R132S) with IC₅₀ values below about 0.5 micromolar. In addition, Compound 1 has displayed relevant levels of activity against multiple clinically relevant, mutated forms of IDH1, of which IDH1-R132H and IDH1-R132C are the most prevalent in hematologic and solid tumor malignancies. However, Compound 1 did not display appreciable activity against wild-type IDH1 or mutated IDH2.

The cellular potency of Compound 1 in suppressing intracellular 2-HG levels was determined in cell lines expressing five different mutated IDH1 proteins found in human cancers (R132H, R132C, R132G, R132L, R132S). The human fibrosarcoma cell line HT-1080 harbors a naturally occurring heterozygous IDH1-R132C mutation. The human colorectal carcinoma cell line HCT 116 is wild type for IDH1, but heterozygous mutations coding for IDH1-R132H or -R132C were introduced by knock-in into the endogenous IDH1 gene locus. Finally, the human astrocytoma cell line U-87 MG is also wild type for IDH1, but expression of five different mutated IDH1 proteins was achieved by stable transfection.

The parental HCT116 line (colon) line does not produce high levels of 2-HG, but the variants used herein (X-MAN HCT-116 lines obtained from Horizon Discovery Ltd.) are engineered to knock-in a heterozygous mutation of either IDH1 R132H or IDH1 R132C. This recapitulates the cellular context in mIDH1 cancer cells where there are both wild type and mutant IDH1 subunits that together form a heterodimer that is responsible for the production of elevated levels of 2-HG. These modified lines can be used as models of IDH1 mutant disease.

Each of these cell lines was treated with Compound 1 for 24 hr, and intracellular 2-HG levels were determined by mass spectroscopy. Compound 1 suppressed 2-HG production in each cell line, with IC₅₀ values ranging from <10 nM to <150 nM. The table below indicates 2-HG IC₅₀ values: below 150 nM (“+”), below 100 nM (“++”), below 50 nM (“+++”) and below 10 nM (“++++”).

| Cell Line | 2-HG IC₅₀ (nM)* |
|---|---|
| HT-1080(IDH1-R132C/+) | ++ |
| HCT 116(IDH1-R132H/+) | +++ |
| HCT 116(IDH1-R132C/+) | + |
| U-87MG/IDH1-R132H | +++ |
| U-87MG/IDH1-R132C | ++ |
| U-87MG/IDH1-R132G | ++++ |
| U-87MG/IDH1-R132L | +++ |
| U-87MG/IDH1-R132S | ++++ |

*Mean +/− SEM where applicable

Compound 1 is therefore a potent inhibitor of a variety of clinically relevant IDH1 mutations in a cellular context.

### Example 3—Compound 1 Potently and Selectively Inhibited 2-HG Production in IDH1 R132H and IDH1 R132C Mutant Enzymes in Biochemical Assays, Compared to Wild Type IDH1 Enzyme and Mutant IDH2 Enzymes

The biochemical potencies of Compound 1 against IDH1 R132H and IDH1 R132C mutants were determined in diaphorase-coupled assays, which measure activity by the determination of the level of remaining co-substrate NADPH after the enzymatic reaction is quenched. Recombinant homodimeric IDH1 R132H or IDH1 R132C mutant enzymes were used in these assays.

In order to evaluate the cellular potency of Compound 1 for other IDH1^{R132} mutations that have been identified in human cancers, IDH1^{R132L}, IDH1^{R132G} and IDH1^{R132S} were expressed in U87MG human glioblastoma cells. Matched IDH1^{R132}H and IDH1^{R132C} lines were also prepared to allow direct comparisons in the same cellular background, as well as to compare the effects observed from the same mutation in different cell lines. As for the HT1080 and HCT-116 cell lines described above, the engineered mIDH1-expressing U87MG cells produced higher concentrations of 2-HG but exhibited a similar growth rate when compared to parental U87MG cells. Inhibition of 2-HG production by Compound 1 in the IDH1^{R132H} and IDH1^{R132C} U87 lines gave IC₅₀ values of 9.0 and 39.0 nM, respectively, which are in close agreement with those seen in the HT1080 and HCT-116 cell lines. In addition, Compound 1 potently inhibited 2-HG production in IDH1^{R132G}, IDH1^{R132S} and IDH1^{R132L} expressing cells with IC₅₀ values of 5.6, 9.2, and 41.7 nM, respectively, suggesting that Compound 1 is a potent inhibitor against a broad spectrum of IDH1^{R132} mutants. In agreement with the previous cell lines studies, Compound 1 was found to have minimal effects on the proliferation of mIDH1 expressing U87MG cells at 10 μM.

Additional results are shown in Table 2, relative to the IC₅₀ value obtained for R132H IDH1 mutated enzyme. Referring to data in Table 2, Compound 1 was found to selectively inhibit the enzymatic activity of the IDH1 R132H and IDH1 R132C mutations with an IC₅₀ value within a factor of about 5 (i.e., the IC₅₀ value measured for IDH1 R132C mutant enzyme was about 5 times higher than the IC₅₀ measured in the IDH1 R132H mutated enzyme). The selectivity of Compound 1 against other IDH isozymes was also tested utilizing diaphorase coupled assays employing either wild-type IDH1 or one of 2 alternate mutated forms of IDH2, namely IDH2 R172K and IDH2 R140Q.

TABLE 2

| Target | Relative Enzymatic IC₅₀ (Average +/− SEM, nM) |
|---|---|
| IDH1 R132H | 1.0 (±6.6%) |
| IDH1 R132C | 5.1 (±6.1%) |
| Wild Type IDH1 | 922 |
| IDH2 R172K | >1,000 |
| IDH2 R1400 | >4,000 (no activity measured) |

Compound 1 had comparatively very weak activity against wild type IDH1 (IC₅₀ value of about 922 times greater than the IC₅₀ value measured for IDH1 R132H). Compound 1 also demonstrated very weak activity against IDH2 R172K that was more than 1,000 greater than the IC₅₀ value measured for IDH1 R132H. Compound 1 did not show any inhibition of IDH2 R140Q up to a concentration of 100 μM. These selectivity data indicate that Compound 1 is a potent and selective inhibitor of IDH1 R132 mutations.

### Example 4—Testing Compound 1 in Mouse Xenograft Models Using HCT 116 Cells with R132C and R132H Mutations

In order to assess the in vivo activity of Compound 1, PK-PD experiments in mice bearing HCT-116 xenografts (derived from Horizon Discovery isogenic cell lines harboring IDH1-R132H and IDH1-R132C knock-in mutations) were used to determine the degree of exposure required to suppress 2-HG levels. Compound 1 was administered to HCT116-IDH1-R132H/+ xenograft bearing female BALB/c Nude mice at three oral doses (12.5, 25, and 50 mg/kg) in 12-hour intervals. Plasma and xenograft tumor samples were collected at 4, 12, and 24 hours post last dose to determine the exposure of Compound 1 in plasma and tumor, as well as to measure the inhibition of IDH1 mutant activity in tumor based on the reduction in levels of 2-HG.

In both IDH1 mutated models, the free concentration of Compound 1 was comparable in plasma and xenograft tumors, and exposures were dose-dependent. In comparison to the vehicle treated group, Compound 1 showed a time and dose-dependent inhibition of 2-HG levels in plasma and in tumor. At the highest dose tested in these studies (50 mg/kg), treatment with Compound 1 inhibited 2-HG levels in the tumor by >90% for up to 24 hours after the last dose in the HCT116-IDH1-R132H/+ xenograft model, and to similar levels for at least 12 hours in the HCT116-IDH1-R132C/+ model. Calculations based upon the percentage of suppression of 2-HG concentration in tumor versus the free drug concentration in tumor gave in vivo IC₅₀ values of 26 nM and 36 nM in the HCT116-IDH1-R132H or HCT116-IDH1-R132C models, respectively. When corrected for unbound levels of Compound 1, there is an excellent correlation in potency among the biochemical assay, cellular assay, and in vivo studies.

TABLE 3

| | |
|---|---|
| mlDH1-R132H | mlDH1-R132C |

| Enzyme | Cell 2-HG | In vivo 2-HG | Enzyme | Cell 2-HG | In vivo 2-HG |
|---|---|---|---|---|---|
| (nM) | (nM) | (nM) | (nM) | (nM) | (nM) |
| 17 | 37 | 26 | 100 | 66 | 36 |

In order to optimize the dosing schedule of Compound 1 to achieve sustained >90% 2-HG inhibition in mIDH1 in vivo, HCT116-IDH1 R132H and HCT116-IDH1 R132C xenograft-bearing mice were treated with Compound 1 at 25 and 50 mg/kg BID (3 doses). The free drug concentration of Compound 1 at 12 hour post final dose is above the in vivo IC₉₀ for all doses, and a greater than 90% reduction of 2-HG levels in tumor were achieved in each case. The free drug concentration decreased to 3-10X the in vivo IC₅₀ at 24 hour post final dose, and Compound 1 showed 80-90% (or greater) inhibition. There was less than 20 nM free drug concentration in tumor at 48 and 72 hours after final dose, and at that point there was less than 50% 2-HG inhibition in tumor samples, consistent with the reduced level of Compound 1.

Briefly, 5×10⁶ HCT-116 IDH1-R132H/+ cells (Horizon Discovery) in PBS was inoculated subcutaneously at the right flank of the 6 weeks old female BALB/c nude mice. When the tumor size reached 360-400 mm³, mice were randomized by tumor volume into nine mice per group. The tumor bearing mice were treated with vehicle (9:1 PEG400:Ethanol) or Compound 1 for three doses with 12 hr dosing interval. The dosing volume was 10 μL/g. The plasma samples and tumor samples were collected at 4, 12 and 24 hr post final dose (N=3 mice per time point) for the subsequent measurement of compound level in plasma and tumor samples and of 2-HG level in the tumor samples by UPLC-MS-MS system.

In a separate dosing example, 5×10⁶ HCT-116 IDH1-R132C/+ cells (Horizon Discovery) in PBS were inoculated subcutaneously at the right flank of the 6-8 weeks old female BALB/c nude mice. When the tumor size reached ˜250 mm³, mice were randomized by tumor volume into nine mice per group. The tumor bearing mice were treated with vehicle (9:1 PEG400:Ethanol) or Compound 1 for six doses with 12 hr dosing interval. The dosing volume was 10 μL/g. The plasma samples and tumor samples were collected at 4, 8 and 12 hr post final dose (N=4 mice per time point) for the subsequent measurement of compound level in plasma and tumor samples and of 2-HG level in the tumor samples by UPLC-MS-MS system.

For each assay, the total concentration of Compound 1 was determined in μM and free Compound 1 concentration was calculated by multiplying the total Compound 1 concentration by 0.043 given that Compound 1 is 95.7% protein binding in mouse plasma. The percentage of 2-HG inhibition in individual tumor sample in the treated groups was normalized to the average of 2-HG concentration in the vehicle group at the corresponding sampling time using the following calculation: % 2-HG inhibition=100*(A−B)/A, where A is the average of 2-HG concentration at the corresponding sampling time, B is the 2-HG concentration in the tumor treated with given dose of Compound 1 and sacked at the given sampling time. The in vivo potency of Compound 1 for suppressing 2-HG in tumor is calculated by plotting the percentage of 2-HG inhibition against corresponding free Compound 1 concentration in tumor and fitting the data with four-parameter logistic equation.

### IDH1-R132H Mutation

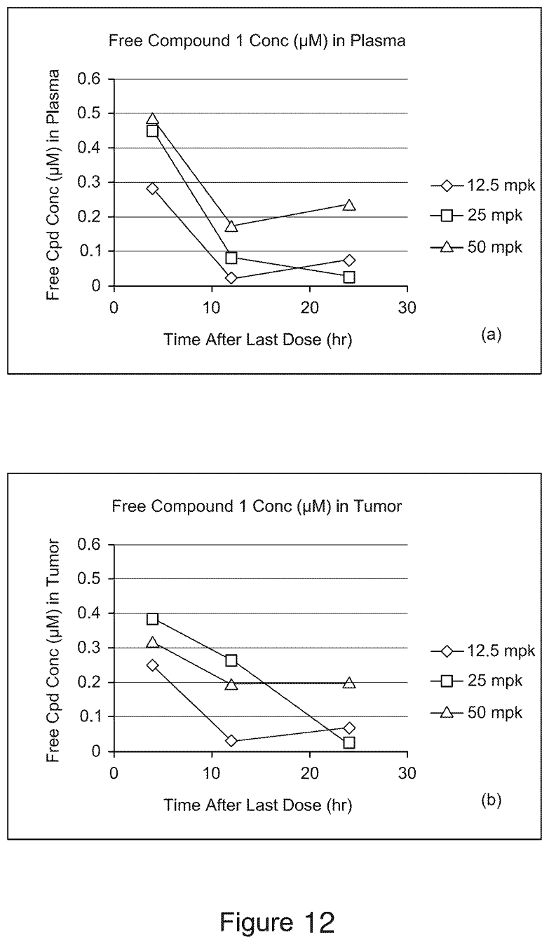

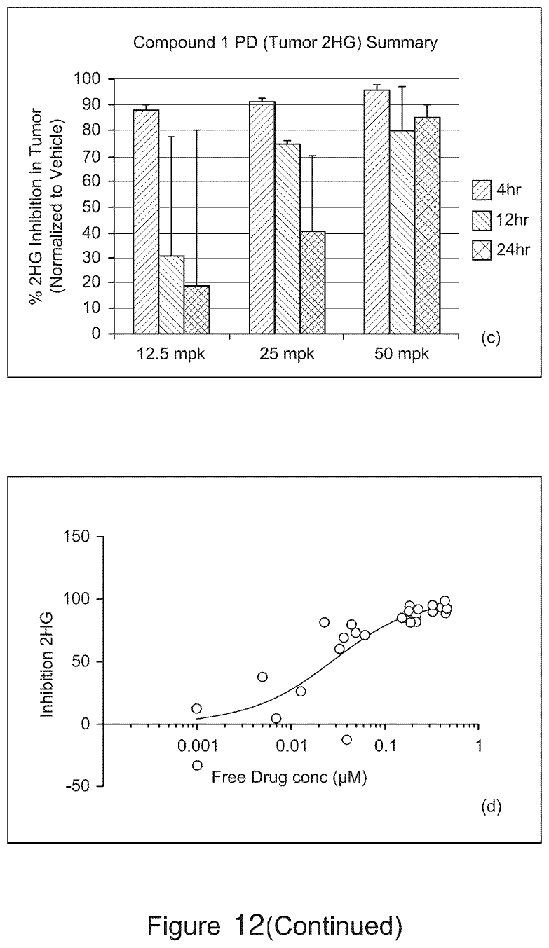

IDH1-R132H mutation resulted in elevation of 2-HG level in hematological and solid cancers. HCT-116 IDH1-R132H/+ xenograft tumor was used to assess the in vivo potency of Compound 1 to suppress 2-HG in tumor lysates. The tumor bearing mice were randomized by tumor size into twelve mice per group. The mice were treated with Compound 1 at 6.25, 12.5, 25, or 50 mg/kg for six doses with dose interval of 12 hr. The plasma and tumor samples were collected at 4, 8, and 12 hr post last dose with four mice per time point. The Compound 1 concentration in plasma and tumor samples was analyzed by LC-MS method (FIG. 12A and FIG. 12B, respectively). The 2-HG level in tumor samples was analyzed by LC-MS method. The percentage of 2-HG suppression in tumor lysate at given dose of Compound 1 was then normalized to 2-HG level in the vehicle control group (FIG. 12C). The dose and time dependent 2-HG inhibition by Compound 1 was observed in this study. The degree of 2-HG inhibition in tumor lysates was correlated with the free drug concentration in the corresponding tumor lysate. The calculated in vivo potency of Compound 1 to suppress 2-HG in tumor was 26.0 nM.

Upon correcting for unbound Compound 1 concentration, there was a good correlation between the enzymatic, cellular 2-HG, and in vivo 2-HG potencies of Compound 1 for IDH1-R132H mutant.

### IDH1-R132C Mutation

IDH1-R132C mutation resulted in elevation of 2-HG level in hematological and solid cancers. HCT-116 IDH1-R132C/+ xenograft tumor was used to assess the in vivo potency of Compound 1 to suppress 2-HG in tumor lysates. The tumor bearing mice were randomized by tumor size into nine mice per group. The mice were treated with Compound 1 at 12.5, 25, or 50 mg/kg for three doses with dose interval of 12 hr. The plasma and tumor samples were collected at 4, 12, and 24 hr post last dose with three mice per time point. The Compound 1 concentration in plasma and tumor samples was analyzed by LC-MS method. The 2-HG level in tumor samples was analyzed by LC-MS method. The percentage of 2-HG suppression in tumor lysate at given dose of Compound 1 was then normalized to 2-HG level in the vehicle control group. The dose and time dependent 2-HG inhibition by Compound 1 was observed in this study. The degree of 2-HG inhibition in tumor lysates was correlated with the free drug concentration in the corresponding tumor lysate. The calculated in vivo potency of Compound 1 to suppress 2-HG in tumor was 36.0 nM.

Upon correcting for unbound Compound 1 concentration, there was a good correlation between the enzymatic, cellular 2-HG, and in vivo 2-HG potencies of Compound 1 for IDH1-R132C mutant.

### Results

Given the role of 2-HG in suppressing normal differentiation of mt-IDH1 cells (Figueria et al., 2010; Saha et al., 2014), it is hypothesized that in order to reverse and maintain this effect, it is necessary to achieve a very high degree of target inhibition on a continuous basis. Therefore, in order to optimize the dosing schedule of Compound 1, it is necessary to achieve sustained >90% 2-HG inhibition in mt-IDH1 in vivo. For the HCT116-IDH1R1^{32H} xenograft assay, the 12 and 24 hour time points were chosen to reflect the compound level and corresponding 2-HG inhibition at the C_{trough} of BID and QD dosing schedules. The 48 and 72 hour time points were selected to investigate whether Compound 1 had long lasting effects on 2-HG inhibition. The free drug concentration of Compound 1 at 12 hour post final dose is above the in vivo IC₉₀ for all doses, and a greater than 90% reduction of 2-HG levels in tumor were achieved in each case. The free drug concentration decreased to 3-10× the in vivo IC₅₀ at 24 hour post final dose, and the compound showed 80-90% inhibition. There was less than 20 nM free drug concentration in tumor at 48 and 72 hours after final dose, and at that point there was less than 50% 2-HG inhibition in tumor samples, consistent with the reduced level of Compound 1. These data support the premise that constant target coverage by a significant margin is required to achieve sustained 2-HG inhibition. This experiment also suggests that a BID schedule is the preferred dosing regimen for Compound 1 in order to continuously achieve >90% 2-HG inhibition. This level of inhibition has recently been correlated to clinical efficacy with AG-221 in mt-IDH2 harboring AML patients (Fan et al., 2014)).

The present disclosure contemplates, among other things, recognition that the total concentration (C_{eff}) of Compound 1 must be above 1652 ng/mL in human patients in order to achieve 90% inhibition of 2-HG and above 2000 ng/mL to achieve greater than 90% inhibition of 2-HG. C_{eff} was determined using assays outlined in this Example. In two separate mouse experiments, HCT-116 IDH1-R132H/+ xenografts and HCT-116 IDH1-R132C/+ xenograft tumor were used to assess the in vivo potency of Compound 1 to suppress 2-HG in tumor lysates. Compound 1 concentration in plasma and tumor samples and 2-HG level in tumor samples was measured. The degree of 2-HG inhibition in tumor lysates was correlated with the free drug concentration in the corresponding tumor lysate (see FIG. 12D). Given the role of 2-HG in suppressing normal differentiation of mt-IDH1 cells (Figueria et al., 2010; Saha et al., 2014), the present disclosure hypothesized that in order to reverse and maintain this effect, it is necessary to achieve a very high degree of target inhibition with Compound 1 on a continuous basis. It was previously proposed that >90% inhibition of 2-HG correlates to clinical efficacy in mt-IDH2 harboring AML patients (FAN, B. et al., Evaluation of the pharmacokinetic/pharmocodynamic (PK/PD) relationships of an oral, selective, first-in-class, potent IDH1 inhibitor, AG-221, from a phase 1 trial in patients with advanced IDH2 mutant positive hematologic malignancies, Blood, 124: 3737, 6 pages (2014)). Using the curve from FIG. 12D, the level of free drug concentration of Compound 1 was determined to be 0.256 μM in order to achieve 90% inhibition of 2-HG.

Using a rapid equilibrium dialysis approach, the plasma protein binding for a human patient was determined to be 94.5%. (Waters, N.J., et al. (2008)). Validation of a rapid equilibrium dialysis approach for the measurement of plasma protein binding. J Pharm Sci 97(10): 4586-95.) Accordingly the total concentration (C_{eff}) can be determined: 0.256/((100−94.5)/100)=4.65 μM=1652 ng/mL.

### Example 5—Case Study of Human Clinical Trial Testing of Compound 1

Patient X is 66 y/o female, diagnosed with AML who initially received induction treatment with high dose cytarabine to which the patient was refractory. Subsequently, the patient enrolled in a clinical trial study, where she was treated with single agent (SA) Compound 1 150 mg BID and achieved a complete remission (CR) after one cycle of treatment (28 days). Patient continued treatment while in CR for 7 additional cycles. Patient then relapsed and discontinued study treatment.

Patient Y is 62 y/o male, diagnosed with FLT3-positive secondary AML (secondary to MDS). Patient received intensive chemotherapy induction with cytarabine and daunorubicin in combination with midostaurin (FLT3 inhibitor) but unfortunately was refractory. He enrolled in a clinical trial study, where he was treated with Compound 1 150 mg BID in combination with azacitidine for a total of 8 cycles (1 cycle=28 days). He achieved complete remission with IDH1 mutation clearance (CRm) by cycle 6 and discontinued study treatment after cycle 8 to undergo bone marrow transplant (HSCT).

Patient Z is a 50 year old diagnosed with grade III IDH1m glioma (anaplastic astrocytoma) previously treated with chemotherapy and radiation according to the applicable standard of care. This patient was subsequently enrolled in the clinical study and treated with Compound 1 at 150 mg twice daily (BID) each day. Following treatment with Compound 1 for 2 cycles (each cycle=28 consecutive days receiving 150 mg Compound 1 BID), by MRI, patient was determined by the investigator to have experienced a partial response by RANO criteria (≥50% decrease in tumor, no new lesions, on stable dose corticosteroids, no progression of measurable disease). After receiving 2 cycles of Compound 1 (150 mg BID), the patient remains on treatment with 150 mg BID Compound 1 per protocol.

3 Patients received a Compound 1 at 100 mg once daily (QD) each day. Blood samples were collected every 28 days for measurement of plasma concentrations of Compound 1 (single agent). Blood was collected at the following times relative to Compound 1 administration:
Cycle 1 Day 1: predose, and postdose at 30 minutes, 1, 2, 4, and 8 hours
Cycle 1 Days 2, 8, 15, and 22: predose
Cycle 2 Day 1: predose, and postdose at 30 minutes, 1, 2, 4, and 8 hours
Cycle 2 Day 2: 24 hours after C2D1 dosing (±2 hours) for patients in dose expansion
Cycle 2 Day 4: predose [72 hours after C2D1 dosing (±4 hours)] for patients in dose expansion only
Cycle 2 Day 15: predose
Cycle 3 and Beyond: Predose on Day 1 of every cycle

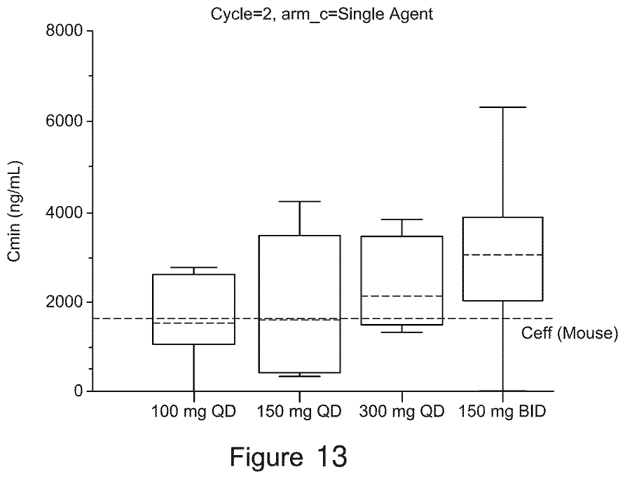

The observed Cₘᵢₙ associated with this case study can be found in FIG. 13.

### Example 6—a Phase 1 Dose Escalation Study of the mIDH-1 Inhibitor, Compound 1, in Patients with AML or Myelodysplastic Syndrome (MDS)

Isocitrate dehydrogenase 1 mutations (m/DH-1) occur in 7-14% of AML patients (“pts.”) and 3% of MDS pts. Compound 1 is a highly potent, selective small molecule inhibitor of mIDH-1 without anticipated CYP or QTc liabilities at the recommended phase 2 dose. Compound 1 was tested in a Phase 1/2 study to evaluate the safety, efficacy, PK, and PD of Compound 1 as a single agent or in combination with azacitidine or cytarabine.

FIG. 5A illustrates the summary of cohorts from a phase 1 study in IDH1m AML and MDS, described in this example. The Phase 1/2 study of Compound 1 was initiated to evaluate the safety, PK/PD, and clinical activity of Compound 1 alone or in combination with azacitidine (AZA) or cytarabine in mIDH-1 AML/MDS pts. In the phase 1 portion of the study, Compound 1 was dose escalated in a 3+3 design to define the maximum tolerated doses (MTDs) or maximum evaluated doses (MEDs) as a single-agent (SA) and in combination with azacitidine (CO) followed by expansion cohorts. Doses evaluated were 150 mg QD (SA, CO), 300 mg QD (SA), and 150 mg BID (SA, CO). Safety was assessed by incidence and severity of treatment emergent AEs (TEAEs) for all pts and efficacy derived by IWG criteria (2003 AML and 2006 MDS) based on investigator assessment for evaluable pts.

FIG. 14 illustrates cycle 1, grade 2 IDH-DS is resolved with dexamethasone and hydroxyurea. C2D1: CRp (1% blasts) to SCT in MLFS, during the clinical trial of this Example.

### Subject Inclusion Criteria can Include:

1. Pathologically proven AML or intermediate, high-risk, or very high risk MDS as defined by the World Health Organization (WHO) criteria or Revised International Prognostic Scoring System (IPSS-R) which is relapsed or refractory (R/R) to standard therapy and/or for which standard therapy is contraindicated or which has not adequately responded to standard therapy, e.g. Compound 1 as a single agent: patient has received no prior IDH1 inhibitor or Compound 1 in combination with Aza: patients are treat-naïve & eligible for Aza, patients can have received IDH1m inhibitor or HMA.
2. Patients must have documented IDH1-R132 gene-mutated disease as evaluated by the site.
3. Good performance status.
4. Good kidney and liver function.
5. Baseline QTcF ≤450 msec.
6. No exclusions for concomitant medications.

### Subject Exclusion Criteria can Include:

1. Patients with symptomatic central nervous system (CNS) metastases or other tumor location (such as spinal cord compression, other compressive mass, uncontrolled painful lesion, bone fracture, etc.) necessitating an urgent therapeutic intervention, palliative care, surgery or radiation therapy.
2. Congestive heart failure (New York Heart Association Class III or IV) or unstable angina pectoris. Previous history of myocardial infarction within 1 year prior to study entry, uncontrolled hypertension or uncontrolled arrhythmias.
3. Pulmonary disease (e.g. COPD, asthma, etc.) that is not controlled (moderate to severe symptoms) with current medication.
4. Active, uncontrolled bacterial, viral, or fungal infections, requiring systemic therapy.

### Treatment/Intervention Plan

Compound 1 was administered as a single agent or in combination with azacitidine or cytarabine. Compound 1 was supplied as 50 mg or 150 mg capsules and was administered per the protocol defined frequency and dose level. Azacitidine was administered per site's standard of care. Cytarabine will be administered per site's standard of care.

The Phase 1 stage of the study was split into 2 distinct parts: a dose escalation part, which will utilize an open-label design of Compound 1 (single agent), or Compound 1+azacitidine (combination agent), or Compound 1+cytarabine (combination agent) administered via one or more intermittent dosing schedules, followed by a dose expansion part. The dose expansion part will enroll patients in up to 5 expansion cohorts, exploring single-agent Compound 1 activity as well as combination activity with azacitidine or cytarabine. Patients may receive only a single dose of study drug (single-agent arm and combination arm) on Cycle 1 Day 1. Following the completion of the relevant Phase 1 cohorts, Phase 2 begins enrollment. Patients are enrolled across 6 different cohorts, examining the effect of Compound 1 (as a single agent) and Compound 1 with azacitidine (combination) on various AML/MDS disease states. Conditions examined include acute myeloid leukemia (also known as acute myelogenous leukemia) and myelodysplastic syndrome.

TABLE 4
Arms and Interventions of Phase 1 Trial.

| Arms | Assigned Interventions |
|---|---|
| Experimental: PH1 Dose Escalation & | Drug: Compound 1 |
| Expansion Compound 1 | Compound 1 is supplied as 50 mg or 150 mg |
| | capsules and is administered per the |
| | protocol defined frequency and dose level |
| Experimental: PH1 Esc. and | Drug: Compound 1 |
| Exp. Compound 1 + Azacitidine | Compound 1 is supplied as 50 mg or 150 mg |
| | capsules and is administered per the |
| Arms | Assigned Interventions |
| | protocol defined frequency and dose level |
| | Drug: Azacitidine (Vidaza) |
| | azacitidine is administered per site’s |
| | standard of care |
| Experimental: PH1 Esc. and | Drug: Compound 1 |
| Exp. Compound 1 + Cytarabine | Compound 1 is supplied as 50 mg or 150 mg |
| | capsules and is administered per the |
| | protocol defined frequency and dose level |
| | Drug: Cytarabine |
| | low-dose cytarabine are administered per |
| | site’s standard of care |
| Experimental: PH2 Cohort 1 | Drug: Compound 1 |
| Compound 1 Single Agent Relapsed | Compound 1 is supplied as 50 mg or 150 mg |
| or Refractory (R/R) AML | capsules and is administered per the |
| | protocol defined frequency and dose level |
| Experimental: PH2 Cohort 2 Compound 1 | Drug: Compound 1 |
| Single Agent | Compound 1 is supplied as 50 mg or 150 mg |
| AML/MDS in morphologic complete | capsules and is administered per the |
| remission or complete remission with | protocol defined frequency and dose level |
| incomplete blood count recovery (CR/CRi) | |
| after cytotoxic-containing induction therapy | |
| with residual IDH-R132 mutation | |
| Experimental: PH2 Cohort 3 Compound 1 | Drug: Compound 1 |
| Single Agent | Compound 1 is supplied as 50 mg or 150 mg |
| R/R AML/MDS, previously treated with an | capsules and is administered per the |
| IDH1 inhibitor | protocol defined frequency and dose level |
| Experimental: PH2 Cohort 4 | Drug: Compound 1 |
| Compound 1 + Azacitidine | Compound 1 is supplied as 50 mg or 150 mg |
| R/R AML/MDS that are naive to prior | capsules and is administered per the |
| hypomethylating therapy and IDH1 inhibitor | protocol defined frequency and dose level |
| therapy | Drug: Azacitidine (Vidaza) |
| | Azacitidine is administered per site’s |
| | standard of care |
| Experimental: PH2 Cohort 5 Compound 1 + | Drug: Compound 1 |
| Azacitidine | Compound 1 is supplied as 50 mg or 150 mg |
| R/R AML/MDS that have inadequately | capsules and is administered per the |
| responded or have progressed immediately | protocol defined frequency and dose level |
| proceeding hypomethylating therapy | Drug: Azacitidine (Vidaza) |
| | Azacitidine is administered per site’s |
| | standard of care |
| Experimental: PH2 Cohort 6 Compound 1 + | Drug: Compound 1 |
| Azacitidine | Compound 1 is supplied as 50 mg or 150 mg |
| Arms | Assigned Interventions |
| R/R AML/MDS that have been previously | capsules and is administered per the |
| treated with single-agent IDH1 inhibitor | protocol defined frequency and dose level |
| therapy as their last therapy prior to study | Drug: Azacitidine (Vidaza) |
| enrollment | azacitidine is administered per site’s |
| | standard of care |

Following the completion of Phase 1, Phase 2 enrollment began. Patients were enrolled across 6 different cohorts, examining the effect of Compound 1 (as a single agent) and Compound 1+azacitidine (combination) on various AML/MDS disease states. The Phase 2 cohorts are summarized in Table 5 below:

TABLE 5

| Cohort | Patient Population | Intervention |
|---|---|---|
| I | Patients with relapsed or | Recommended phase II dose |
| | refractory (R/R) AML | (“RP2D”) of Compound 1 as a |
| | | single-agent |
| II | Patients with AML/ MDS in | RP2D of Compound 1 as a |
| | morphologic complete | single-agent |
| | remission or complete | |
| | remission with incomplete | |
| | blood count recovery (CR/CRi) | |
| | after cytotoxic-containing | |
| | induction therapy with | |
| | residual IDH-R132 mutation | |
| III | Patients with R/R AML/MDS, | RP2D of Compound 1 as a |
| | previously treated with an | single-agent |
| | IDH1 inhibitor | |
| IV | Patients with R/R AML/MDS | RP2D of Compound 1 in |
| | that are naive to prior | combination with azacitidine |
| | hypomethylating therapy and | |
| | IDH1 inhibitor therapy | |
| V | Patients with R/R AML/MDS | RP2D of Compound 1 in |
| | that have inadequately | combination with azacitidine |
| | responded or have progressed | |
| | immediately preceding | |
| | hypomethylating therapy | |
| VI | Patients with R/R AML/MDS | RP2D of Compound 1 in |
| | that have been previously | combination with azacitidine |
| | treated with single-agent | |
| | IDH1 inhibitor therapy as their | |
| | last therapy prior to study | |
| | enrollment | |

### Primary Outcome Measures

The outcome of the study can be evaluated using the following criteria:
1. Maximum Tolerated Doses (MTDs) or Maximum Evaluated Doses (MEDs) [Phase 1]. Time Frame: Within first 4 weeks of treatment.
2. Number of Participants with a Dose Limiting Toxicity (DLT) [Phase 1]. Time Frame: Within first 4 weeks of treatment. DLT Criteria can include:
   ≥Gr 3 non-hematologic toxicity or laboratory finding
   Gr 4 hematologic toxicity by Day 42 in absence of disease
   Inability to tolerate at least 75% of Cycle 1 treatment
3. Doses recommended for future studies [Phase 1]. Time Frame: Within first 4 weeks of treatment.
4. Complete Response (CR, CRi, MLFS, Marrow CR) Rate of Compound 1 as a single-agent or in combination with azacitidine in patients with AML/MDS [Phase 2]. Time Frame: As per IWG Response Assessment Guidelines for AML and MDS based on investigator's assessment through study completion, e.g. modified IWG AML 2003/MDS 2006.

### Secondary Outcome Measures

The outcome of the study can also be evaluated using the following criteria:
1. Area under the plasma concentration versus time curve (AUC) [Phase 1 and Phase 2]. Time Frame: Blood samples for PK analysis collected at multiple visits during the first 60 days of treatment and on day 1 of all cycles following the first 30 days.
2. Peak Plasma Concentration (Cmax) [Phase 1 and Phase 2]. Time Frame: Blood samples for PK analysis collected at multiple visits during the first 60 days of treatment and on day 1 of all cycles following the first 30 days.
3. Time of peak plasma concentration (Tmax) [Phase 1 and Phase 2]. Time Frame: Blood samples for PK analysis collected at multiple visits during the first 60 days of treatment and on day 1 of all cycles following the first 30 days.
4. Time for half of the drug to be absent in blood stream following dose (T 1/2) [Phase 1 and Phase 2]. Time Frame: Blood samples for PK analysis collected at multiple visits during the first 60 days of treatment and on day 1 of all cycles following the first 30 days.
5. Rate at which drug is removed from blood stream (CL/F) [Phase 1 and Phase 2]. Time Frame: Blood samples for PK analysis collected at multiple visits during the first 60 days of treatment and on day 1 of all cycles following the first 30 days.
6. Rate of drug distribution within the blood stream (Vd/F) [Phase 1 and Phase 2]. Time Frame: Blood samples for PK analysis collected at multiple visits during the first 60 days of treatment and on day 1 of all cycles following the first 30 days.
7. Reduction of 2-HG levels in plasma [Phase 1 and Phase 2]. Time Frame: Blood samples for PK/PD analysis collected at multiple visits during the first 60 days of treatment and on day 1 of all cycles following the first 30 days.
8. Evidence of antileukemic or antimyelodysplastic activity of Compound 1 as determined by complete response (CR), CRi (complete remission with incomplete hematologic recovery), morphologic leukemia-free state (MLFS), Marrow CR, partial remission (PR), and stable disease (SD) as a single-agent or in combination with azacitidine or cytarabine [Phase 1]. Time Frame: As per IWG Response Assessment Guidelines for AML and MDS based on investigator's assessment through study completion.
9. Incidence and severity of adverse events, clinical laboratory abnormalities, and changes in ECG parameters as assessed by CTCAE v4.0 as a single-agent or in combination with azacitidine [Phase 2]. Time Frame: Safety will be assessed from time of first dose through 28 days post last dose.
10. Additional measures of antileukemic or antimyelodysplastic activity as determined by CRh, Overall Response (OR), and Stable Disease of Compound 1 alone or in combination with azacitidine [Phase 2]. Time Frame: As per IWG Response Assessment Guidelines for AML and MDS based on investigator's assessment through study completion.
11. Time to Response (TTR) [Phase 2]. Time Frame: From first dose of study drug through time of first response by blood recovery count.
12. Duration of Response (DOR) [Phase 2]. Time Frame: From time of first response by blood recovery count through relapse.
13. Event-Free Survival (EFS) [Phase 2]. Time Frame: From time of entry on study through progression.
14. Overall Survival (OS) [Phase 2]. Time Frame: From time of entry on study through death or date last known alive at end of follow-up.

### Disease History and Baseline Characteristics of Exemplary Participants

A summary the disease history and baseline characteristics of exemplary participants is shown in Table 6.

TABLE 6

| | Compund 1 | Compund 1 + |
|---|---|---|
| Characteristic | n = 31 | Azacitidine n = 41 |
| Age, median (range), years | 71 (35-87) | 66 (31-88) |
| Female, % | 52 | 51 |
| ECOG PS-0/1/2, % | 25/65/10 | 32/51/17 |
| IDH1 mutation type R132-, n | C(13)/H(9)/S(4)/ | C(22)/H(12)/S(4)/G(2)/ |
| | G(4)/L(1) | Others(1*) |
| AML, n | 25 | 35 |
| Relapsed (>12 mo) | 14 | 1 |
| Relapsed (≤12 mo) | 4 | 10 |
| Refractory | 8 | 15 |
| Treatment- naïve | 3 | 9 |
| Secondary AML | 3 | 12 |
| t-AML | 1 | 1 |
| Prior regimens, median (range) | 2 (0-10) | 3 (0-6) |
| Prior HMA/ | no prior IDHm inhibitor | 6/3/1 |
| IDHm inhibitor/Both | | |
| MDS, n | 6 | 6 |
| Relapsed/Refractory | 4 | 2 |
| Treatment naive | 2 | 4 |
| Prior regimens, median (range) | 1 (0-4) | 1 (0-4) |
| Prior HMA/ | no prior IDHm inhibitor | 2/0/0 |
| IDHm inhibitor/Both | | |

*One patient with a different non-R132 mutation variant

### Results

In the human clinical trial described in this Example, when Compound 1 was administered as a single agent, it provided a 41% overall response rate (ORR) and 27% CR/CRh in R/R AML. Combination of Compound 1 with azacitidine provided a 46% ORR and 16% CR/CRh in R/R AML.

At the data cutoff, 35 pts with a median of 2 prior regimens (range 1-9) had received Compound 1 in dose-escalation, including 31 single-agent (SA) and 41 azacitidine combination (CO) pts. Steady-state exposure that exceeded the target IC₉₀ for m/DH-1 was achieved at 150 mg BID, resulting in a reduction of 2-HG to normal levels in the majority of pts. Furthermore, administration of Compound 1, both as SA and in CO at 150 mg BID, enabled all pts to achieve the target Css that exceeded the IC₉₀ for mIDH-1 while staying below the exposures projected from the monkey toxicology studies that could be associated with QT prolongation. PK data were available through Cycle 10; steady state plasma drug levels were maintained at target Css over the evaluated period. A reduction of 2-HG was observed across all dose levels, with pts receiving 150 mg BID having a median within the normal 2-HG limits.

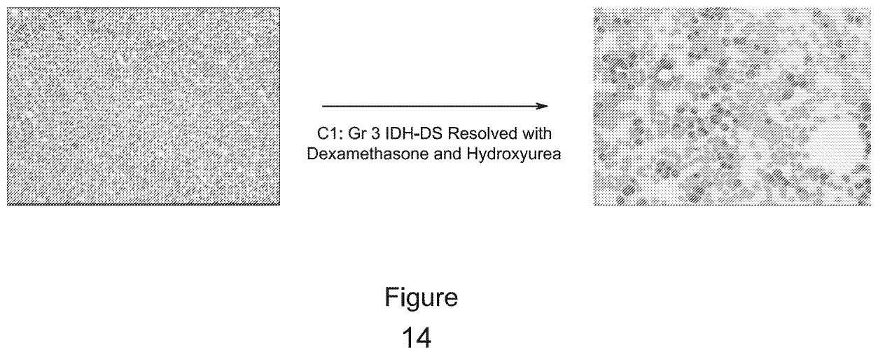

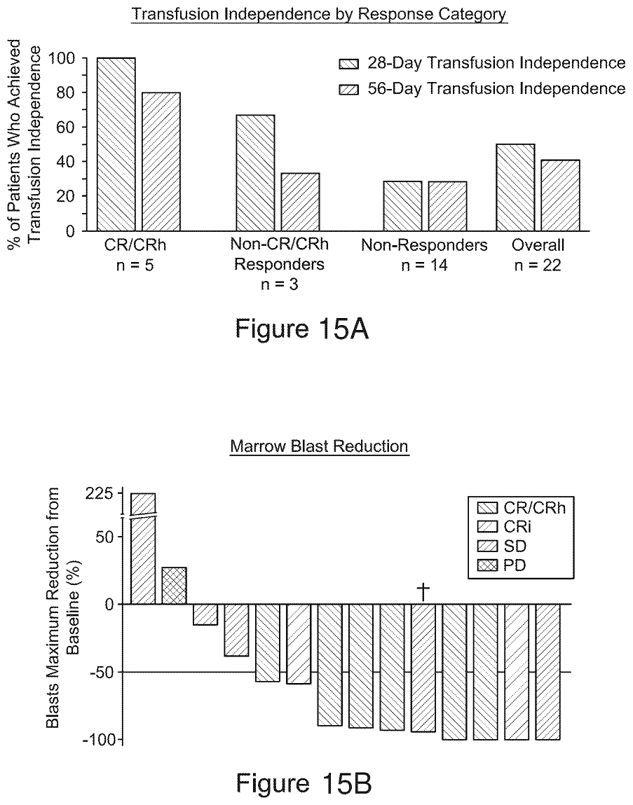

As shown in FIG. 15A, transfusion independence (platelets and/or red blood cells) was observed in all response categories in AML patients treated with Compound 1 as a single agent who were transfusion-dependent at baseline. As shown in FIG. 15B, significant reduction in bone marrow blast content was observed in patients with a CR/CRi response per IWG response criteria. Reductions in bone marrow blast content were also observed in patients in the absence of IWG response (stable disease) including 1 subject (denoted with t in FIG. 15B) with bone marrow blast count <5% but with blasts present in peripheral blood.

The responses per Investigator assessment per modified IWG are summarized in Table 7:

TABLE 7
Responses to Compound 1 as a single agent.

| | R/R AML | All AML & MDS |
|---|---|---|
| Response | N = 22 | N = 31 |
| ORR, n (%)* | 9 (41) | 11 (35) |
| [95% CI] | [21, 64] | [19, 55] |
| CR, n (%) | 4 (18) | 5 (16) |
| CRh, n (%) | 2 (9) | 2 (6) |
| CRi, n (%) | 3 (14) | 4 (13) |
| PR, n (%) | 0 | 0 |
| MLFS, n (%) | 0 | 0 |
| SD, n (%) | 5 (23) | 11 (35) |
| PD, n (%) | 1 (5) | 1 (3) |
| NE, n (%) | 7 (32) | 8 (26) |

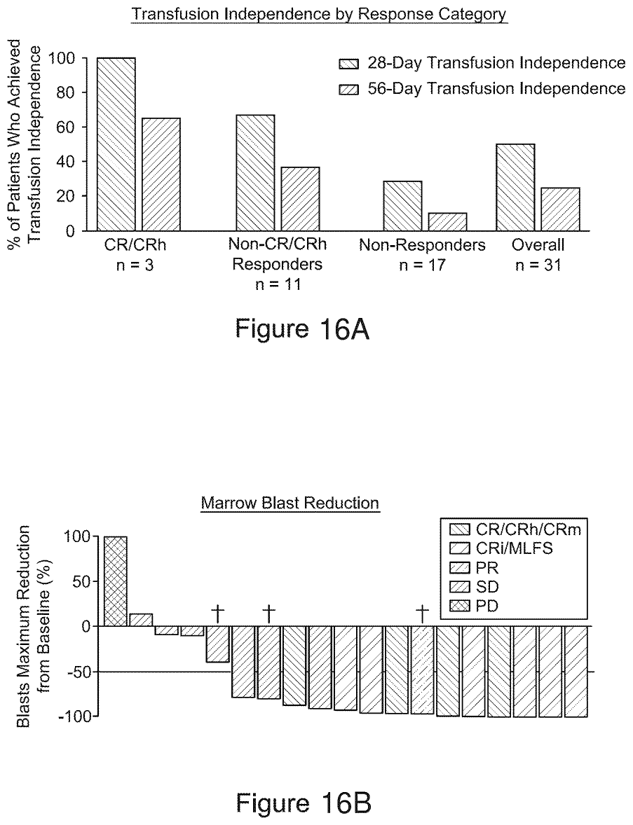

As shown in FIG. 16A, transfusion independence (platelets and/or red blood cells) was observed in all response categories in AML patients treated with Compound 1 and azacitidine who were transfusion-dependent at baseline. As shown in FIG. 16B, reduction in bone marrow blast content was observed in patients with a CR/CRi response per IWG response criteria. Reductions in bone marrow blast content were also observed in patients in the absence of IWG response (stable disease) including 1 subject (denoted with t in FIG. 16B) with bone marrow blast count <5% but with blasts present in peripheral blood. Marrow blast reduction >50% in patients with SD indicates clinical activity in the absence of IWG response.

The responses per Investigator assessment per modified IWG are summarized in Table 8.

TABLE 8
Responses to Compound 1 + azacitidine.

| | R/R AML | TN AML | All AML & MDS |
|---|---|---|---|
| Response | N = 26 | N = 9 | N = 41 |
| ORR, n (%)* | 12 (46) | 7 (78) | 22 (54) |
| [95% CI] | [27, 67] | [40, 971 | [37, 691 |
| CR, n (%) | 3 (12) | 2 (22) | 8 (20) |
| CRh, n (%) | 1 (4) | 0 | 1 (2) |
| CRi, n (%) | 5 (19) | 4 (44) | 9 (22) |
| PR, n (%) | 1 (4) | 1 (11) | 2 (5) |
| MLFS, n (%) | 2 (8) | 0 | 2 (5) |
| SD, n (%) | 11 (42) | 1 (11) | 14 (34) |
| PD, n (%) | 1 (4) | 0 | 1 (2) |
| NE, n (%) | 2 (8) | 1 (11) | 4(10) |

### Exemplary Patient Disposition

A summary of the patient disposition is shown in Table 9.

TABLE 9

| | Compound 1 | Compound 1 + |
|---|---|---|
| Characteristic | n = 31 (%) | Azacitidine n = 27 (%) |
| Patients enrolled, n | 31 | 27 |
| Patients treated | 31 (100) | 26 (96) |
| Median months on treatment | 3 (<1-20 m) | 3 (<1-12 m) |
| (range) | | |
| Patients on treatment | 13 (42) | 13 (50) |
| Discontinued treatment | 18 (58) | 13 (50) |

Reason for Discontinuation

| | | |
|---|---|---|
| Death | 6 | 4 |
| Progressive disease | 9 | 6 |
| Transplant | 4 | 6 |
| Other * | 3 | 3 |
| Investigator decision | 2 | 5 |
| Withdrawal of consent | 1 | 0 |
| Adverse event | 3 | 2 |

*SA: 3 patients discontinued due to resistant disease/ lack of response
Combo: 1 patient discontinued for refusal of treatment, 1 patient to enter hospice, and 1 patient with no reason for discontinuation given.

### Treatment-Emergent Adverse Events (TEAEs) ≥15% all Grades

A summary of TEAEs can be found in Tables 10 and 11.

TABLE 10

| | Compound 1, n = 31 (Single Agent) | |
|---|---|---|
| AE Preferred Term | All Grade (%) | Grade 3/4 (%) |
| Fatigue | 13 (42) | 1 (3) |
| Nausea | 13 (42) | 0 |
| Pyrexia | 10 (32) | 2 (7) |
| Pneumonia | 7 (23) | 5 (16) |
| Vomiting | 7 (23) | 0 |
| Dyspnea | 7 (23) | 0 |
| Diarrhea | 6 (19) | 0 |
| Dizziness | 6 (19) | 1 (3) |
| Decreased Appetite | 6 (19) | 0 |
| Constipation | 6 (19) | 1 (3) |
| Hypokalemia | 6 (19) | 2 (7) |
| AST | 5 (16) | 2 (7) |
| Abdominal | 5 (16) | 0 |
| distention | | |
| Epistaxis | 5 (16) | 1 (3) |
| Headache | 5 (16) | 0 |
| Thrombocytopenia¹ | 9 (29) | 9 (29) |
| Leukocytosis | 8 (26) | 4 (13) |
| Anemia | 7 (23) | 7 (23) |
| Febrile neutropenia | 7 (23) | 7 (23) |

¹Includes preferred term of platelet count decreased

TABLE 11

| | Compound 1, n = 41 (Cpd 1 + AZA) | |
|---|---|---|
| AE Preferred Term | All Grade (%) | Grade 3/4 (%) |
| Nausea | 20 (49) | 3 (7) |
| Constipation | 20 (49) | 1 (2) |
| Hypokalemia | 15 (37) | 4 (10) |
| Fatigue | 15 (37) | 7 (17) |
| Diarrhea | 13 (32) | 2 (5) |
| Vomiting | 9 (22) | 0 |
| Headache | 9 (22) | 1 (2) |
| Decreased Appetite | 8 (20) | 1 (2) |
| Cough | 7 (17) | 1 (2) |
| Pneumonia | 7 (17) | 6 (15) |
| Pruritus | 7 (17) | 0 |
| Dysgeusia | 7 (17) | 0 |
| Creatinine | 7 (17) | 1 (2) |
| increased | | |
| Dizziness | 6 (15) | 1 (2) |
| Asthenia | 6 (15) | 2 (5) |
| Abdominal Pain | 6 (15) | 1 (2) |
| Thrombocytopenia¹ | 15 (37) | 12 (29) |
| Febrile neutropenia | 12 (29) | 12 (29) |
| Neutropenia² | 10 (24) | 7 (17) |
| Anemia | 9 (22) | 7 (17) |
| Leukocytosis | 9 (22) | 6 (15) |
| Leukpenia | 7 (17) | 5 (12) |

¹Includes preferred term of platelet count decreased
²lncludes preferred term of neutrophil count decreased

### Adverse Events (AE) of Interest and Deaths

AEs were assessed per National Cancer Institute's Common Terminology Criteria for Adverse Events (NCI CTCAE), version 4.03.

No DLTs were observed in dose escalation.

### Compound 1 as Single Agent

4 (13%) patients receiving Compound 1 as a single agent exhibited IDH-DS; all resolved with treatment interruption, dexamethasone, hydroxyurea and supportive care and then resumed treatment with Compound 1.

The QTcF maximum change from baseline for patients treated with Compound 1 as a single agent is reported in Table 12.

TABLE 12

| | Maximum Post-Baseline Value | | | | |
|---|---|---|---|---|---|
| | | | >480-≤500 | | >60 msec Δ |
| Treatment | Baseline | ≤480 msec | msec | >500 msec | from BL |
| Group | Value | n (%) | n (%) | n (%) | n (%) |
| Total | ≤480 msec | 27 (90) | 0 | 0 | 1 (3.3)^{‡} |
| SA (N = 30) | | | | | |
| Compound 1 | 480-≤500 | 0 | 1 (3.3)^{†} | 0 | 0 |
| | msec | | | | |
| | >500 msec | 0 | 0 | 1 (3.3) | 0 |
| | Total | 27 (90) | 1 (3.3) | 1 (3.3) | 1 (3.3) |

2 patients with BBB (t in Table 12) enrolled with QTc readings above normal at baseline remained stable within the same QTc range on treatment. One patient (†in Table 12) had an increase of >60 msec but remained within the normal limits (<450 msec).

9 patients receiving Compound 1 as a single agent died within 30 days of last dose due to AEs unrelated to treatment with Compound 1. The AEs unrelated to treatment with Compound 1 which resulted in death are summarized below:
Progressive Disease (PD) (n=3)
Central Nervous System (CNS) Events (n=2)
Sepsis (n=1)
Cardiac Arrest (n=1)
Multiorgan failure (n=1)
toxicity to HSCT regime (n=1)
   Compound 1 in Combination with Azacitidine

The QTcF maximum change from baseline (BL) for patients treated with Compound 1 and azacitidine in combination is reported in Table 13.

TABLE 13

| | Maximum Post-Baseline Value | | | | |
|---|---|---|---|---|---|
| | | | >480-≤500 | | >60 msec Δ |
| Treatment | Baseline | ≤480 msec | msec | >500 msec | from BL |
| Group | Value | n (%) | n (%) | n (%) | n (%) |
| Total | ≤480 msec | 37 (90.0) | 2 (5) | 2 (5) | 4 (10) |
| Compound 1 + | | | | | |
| AZA 75 | >480-≤500 | 0 | 0 | 0 | 0 |
| mg/m² (N = 41) | msec | | | | |
| | >500 msec | 0 | 0 | 0 | 0 |
| | Total | 37 (90.0) | 2 (5) | 2 (5) | 4 (10) |

Referring to Table 13, 4 patients with >60 msec which increased from BL included the 2 patients with suspect concomitant medications, 1 patient developed G1 prolongation and 1 patient remained within normal limits. Among the patients with QTcF <480 msec at BL, 2 had values of 480-500 msec, and 2 had values >500 msec. 1 of each group (including 1 with pacemaker) had prolonged QTcF before treatment start. The other 2 had transient prolongation that normalized once suspect concomitant medications discontinued 2 AEs of QTcF prolongation reported on study (G2 and G3). These were transient and patients resumed treatment once suspect concomitant medications discontinued.

7 patients receiving Compound 1 and azacitidine died within 30 days of last dose due to AEs unrelated to treatment with Compound 1. The AEs unrelated to treatment with Compound 1 which resulted in death are summarized below:
Progressive Disease (PD) (n=3)
Central Nervous System (CNS) Events (n=2)
Pneumonia (n=1)
Gastrointestinal fistula (n=1)

The present disclosure includes, among other things, the novel understanding that administration of 300 mg of Compound 1 (e.g., 150 mg BID or 300 mg QD) to a patient or population of patients results in a sustained therapeutically effective trough blood plasma concentration (C.). Such a C. of Compound 1 resulted in a durable reduction in 2-HG plasma level over the course of at least 6 treatment cycles.

As outlined in this Example, the concentration total plasma concentration of Compound 1 and the plasma concentration of 2-HG was measured in the blood of patients receiving one of three different dose and dose intervals: 150 mg QD, 300 mg QD or 150 mg BID (either receiving Compound 1 as a single agent or in combination with azacitidine as described in the clinical trial of this Example, in each category). The 2-HG levels were measured prior to administration of Compound 1, and then measured after administration of Compound 1 up to cycle 2, day 1 after first receiving Compound 1 (as the solid form obtained from Example 1).

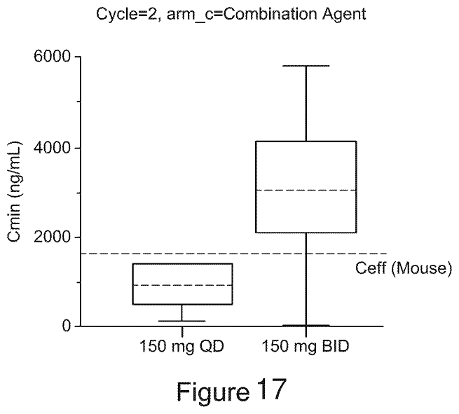

As shown in FIG. 13 and FIG. 17, the administration of Compound 1 at 150 mg BID resulted in a trough blood plasma concentration above 1,652 ng/mL after cycle 2 of a 28-day treatment cycle as both a single agent and in combination with azacitidine.

As shown in FIG. 2A-2E, the plasma exposures (steady state blood plasma concentration) of Compound 1 were durable (i.e., sustained) throughout at least a 6 cycle treatment duration.

### Example 7—Pharmaceutical Product Comprising Olutasidenib

This Example describes a pharmaceutical product comprising olutasidenib (i.e., Compound 1) as the only active moiety and various inactive components (e.g., excipients) (“PRODUCT”) that is useful for the treatment of certain forms of cancer.

Olutasidenib (i.e., Compound 1) has a molecular formula of C₁₈H₁₅ClN₄O₂, a molecular weight of 354.8, and the following structure:

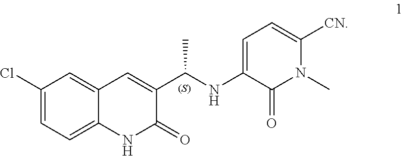

Olutasidenib can also be identified by the following chemical names:
2-Pyridinecarbonitrile, 5-[[(15)-1-(6-chloro-1,2-dihydro-2-oxo-3-quinolinyl)ethyl]amino]-1,6-dihydro-1-methyl-6-oxo-;
5-{[(1S)-1-(6-chloro-2-oxo-1,2-dihydroquinolin-3-yl)ethyl]amino}-1-methyl-6-oxo-1,6-dihydropyridine-2-carbonitrile; and
(S)-5-((1-(6-chloro-2-oxo-1,2-dihydroquinolin-3-yl)ethyl)amino)-1-methyl-6-oxo-1,6-dihydropyridine-2-carbonitrile.

Olutasidenib also has the following identifiers:
Code designation: FT-2102
CAS Registry Number: 1887014-12-1
UNII: 0T41MT8S5Z
WHO Number: 11036

An oral unit dosage form comprising a pharmaceutically acceptable solid form of olutasidenib (e.g., as obtained from Step 7 in Example 1) can be formulated as a drug product with various inactive components as excipients (e.g., as a tablet or capsule) (in this Example, referred to as the “PRODUCT”). Each drug product excipient in PRODUCT meets the requirements of the respective current United States Pharmacopeia (USP) or National Formulary (NF) monograph. The capsule shells can comprise gelatin and about 2.9% w/w of titanium dioxide (E171). Preferably, each oral unit dosage form comprises a total of 50 mg or 150 mg of the pharmaceutically acceptable form of olutasidenib (e.g., micronized crystalline olutasidenib) combined (e.g., at 30-50% w/w) as the only active moiety with pharmaceutically acceptable excipients such as a filler (e.g., AVICEL PH101 @50 micron, AVICEL PH102 @100 micron), a disintegrant (e.g., Ac-Di-Sol), optionally one or more compounds as a lubricant (e.g., magnesium stearate), a glidant/anti-adherent, and/or anti-static (e.g., colloidal silicon dioxide). The excipients can form about 50-70% by weight of the pharmaceutical oral unit dosage form. In one example, a capsule or tablet comprises a total of about 33% of olutasidenib, with the remaining weight of the capsule or tablet is formed from excipients and/or capsule material (e.g., a gelatin). Alternatively, the PRODUCT can be provide as tablet for oral administration. Each tablet can contain the following inactive ingredients: colloidal silicon dioxide, croscarmellose sodium, hypromellose acetate succinate, magnesium stearate, microcrystalline cellulose, and sodium lauryl sulfate. The tablet coating can include FD&C blue #2, hypromellose, lactose monohydrate, titanium dioxide, and/or triacetin.

In one example, PRODUCT is indicated for the treatment of adult patients with relapsed or refractory acute myeloid leukemia (AML) with a susceptible isocitrate dehydrogenase-1 (IDH1) mutation as detected by an FDA-approved test. The PRODUCT is indicated for the treatment of adult patients with relapsed or refractory acute myeloid leukemia (AML) with a susceptible isocitrate dehydrogenase-1 (IDH1) mutation as detected by an FDA-approved test. The PRODUCT is administered in a total of about 300 mg of Compound 1 per day to the patient, preferably 150 mg of olutasidenib twice per day. When appropriate, the dose of the PRODUCT (i.e., amount of olutasidenib) can be decreased to 150 mg once per day or 100 mg twice per day.

Optionally, patients are selected for the treatment of AML with PRODUCT based on the presence of IDH1 mutations in the blood or bone marrow. Patients without IDH1 mutations at diagnosis should be retested at relapse because a mutation in IDH1 may emerge during treatment and at relapse. Information on FDA-approved tests for the detection of IDH1 mutations in AML is available at www.fda.gov/CompanionDiagnostics.

The recommended dose of PRODUCT is 150 mg taken orally twice daily until disease progression or unacceptable toxicity, although dose reductions to 100 mg taken orally twice daily or 150 mg taken orally once daily can also be appropriate for some patients. For patients without disease progression or unacceptable toxicity, treat for a minimum of 6 months to allow time for clinical response.

### Example 8—Pharmaceutical Product and Companion Diagnostic for Identifying and Treating AML Patients Having an IDH1 Mutation

AML is a rapidly progressing cancer that forms in the bone marrow and results in an increased number of abnormal white blood cells in the bloodstream and bone marrow. The National Cancer Institute at the National Institutes of Health estimates that approximately 19,520 people will be diagnosed with AML this year; approximately 10,670 patients with AML will die of the disease in 2018.

Example 7 describes a pharmaceutical product comprising Compound 1 as the only active moiety and various inactive components (e.g., excipients) that is useful for the treatment of certain forms of cancer (“PRODUCT”, e.g., the PRODUCT described in Example 7). PRODUCT comprises an isocitrate dehydrogenase-1 inhibitor (Compound 1) that can decrease abnormal production of the oncometabolite 2-hydroxyglutarate (2-HG), leading to differentiation of malignant cells. If an IDH1 mutation is detected in blood or bone marrow samples using an FDA-approved test, a patient may be eligible for treatment with PRODUCT. One example of such a FDA-approved test is the IDH1 Assay of this Example, a companion diagnostic that can be used to detect this mutation. The IDH1 Assay of this Example is an in vitro polymerase chain reaction (PCR) assay for the qualitative detection of single nucleotide variants (SNVs) coding five IDH1 R132 mutations (R132C, R132H, R132G, R132S, and R132L) in DNA extracted from human blood (EDTA) or bone marrow (EDTA). The IDH1 Assay of this Example is for use with a real-time PCR system.

The IDH1 Assay of this Example is indicated as an aid in identifying acute myeloid leukemia (AML) patients with an isocitrate dehydrogenase-1 (IDH1) mutation for treatment with PRODUCT (Compound 1). This test is for prescription use only.

The IDH1 Assay of this Example detects single nucleotide variants (SNVs) coding five IDH1 mutations (R132C, R132H, R132G, R132S, and R132L) by using PCR technology with homogeneous real-time fluorescence detection. The assay uses human blood or bone marrow aspirate specimens and reports a qualitative result. Table 14 lists the IDH1 mutations detected by the IDH1 Assay of this Example.

TABLE 14
Mutations Detected by the IDH1 Assay of this Example

| Codon | IDH1 Mutation | SNV |
|---|---|---|
| R132 | R132C | TGT |
| | R132H | CAT |
| | R132G | GGT |
| | R132S | AGT |
| | R132L | CTT |

The IDH1 Assay of this Example is a FDA-approved IDH1 companion diagnostic sold under the trade name Abbott RealTime IDH1 (PMA Applicant: Abbott Molecular Inc., 1300 E. Touhy Avenue, Des Plaines, IL 60018; FDA Approval Date: Jul. 20, 2018). It will be appreciated that further details on using IDH1 Assay of this Example are available in product literature and instruction manuals accompanying the assay and/or the real-time PCR system.

### Biological Principles of the Procedure

The IDH1 Assay of this Example consists of two kits:
IDH1 amplification reagent kit
IDH1 control kit

Specimens for the IDH1 Assay of this Example are processed manually using reagents (e.g., lysis buffer containing guanidine isothiocyanate, magnetic microparticles, wash buffers, and/or elution buffer) to isolate and purify sample DNA. The amplification reagents are combined into two amplification master mixes. The purified DNA sample is combined with the master mixes in a 96-well optical reaction plate, and the plate is transferred to a real-time PCR instrument for amplification and detection of IDH1 mutations. The specimen result is automatically reported on a real-time PCR workstation at run completion. Assay controls are included within each run and are processed through DNA extraction, amplification, and detection steps of the assay to assess run validity.

Software parameters specific to the IDH1 Assay of this Example are contained in an assay application specification file, which is loaded onto a real-time PCR instrument by using a CD-ROM disk.

### DNA Extraction

The purpose of DNA extraction is to isolate and purify genomic DNA from EDTA preserved blood or bone marrow aspirate specimens to make it accessible for amplification and to remove potential inhibitors of amplification. This process is accomplished by using magnetic particle technology to isolate and purify DNA. During the DNA extraction procedure, cells are lysed at an elevated temperature in a lysis buffer containing guanidine isothiocyanate. DNA is captured on magnetic microparticles, and inhibitors are removed by performing a series of washes with wash buffers. The bound DNA is eluted from the microparticles with elution buffer and is ready for PCR amplification.

### Reagent Preparation and Reaction Plate Assembly

IDH1 oligonucleotide reagents (Oligonucleotide Reagent 1 and Oligonucleotide Reagent 2) are each manually combined with a DNA polymerase and activation reagent to create 2 unique master mixes. These master mixes are added to 2 separate wells of a 96-well optical reaction plate along with aliquots of the extracted DNA sample. After manual application of an optical adhesive cover, the plate is transferred to a real-time PCR instrument.

### Amplification/Detection

Each master mix is designed to amplify and detect 2 or 3 IDH1 amino acid mutations (codon with mutant nucleotide underlined). Oligonucleotide 1 master mix amplifies and detects R132C (TGT) and R132H (CAT). Oligonucleotide 2 master mix amplifies and detects R132G (GGT), R132S (AGT), and R132L (CTT). Refer to Table 15. In addition, both master mixes are designed to amplify and detect a region of the IDH1 gene outside of codon 132, which serves as an endogenous internal control (IC).

TABLE 15
IDH Mutation Detected by Each Master Mix

| Master Mix | IDH1 Mutation | SNV |
|---|---|---|
| Oligonucleotide 1 | R132C | TGT |
| | R132H | CAT |
| Oligonucleotide 2 | R132G | GGT |
| | R132S | AGT |
| | R132L | CTT |

During the amplification reaction on a real-time PCR instrument, the target DNA is amplified by DNA polymerase in the presence of primers, deoxyribonucleoside triphosphates (dNTPs), and magnesium chloride (MgCl₂). The DNA polymerase used in the assay is a thermophilic enzyme that has been chemically modified, rendering it inactive (e.g., inactive at ambient temperature).

During the amplification reaction of the IDH1 Assay of this Example, DNA polymerase is first activated at a high temperature. During each subsequent round of thermal cycling, a high temperature is used to melt double-stranded DNA strands, followed by a low temperature where primers anneal to their respective targets and are extended to generate double-stranded DNA products. Exponential amplification of the products is achieved through repeated cycling between high and low temperatures. Amplification of IDH1 mutation and IC targets takes place simultaneously in the same PCR well.

IDH1 products are detected during the annealing/extension step by measuring the real-time fluorescence signals of the IDH1 mutation and IC-specific probes, respectively. The IDH1 mutation and IC-specific probes are labeled with different fluorophores, allowing their signals to be distinguishable in a single PCR well.

### Assay Protocol

The IDH1 Assay protocol includes the following steps:
A. Manual preparation (i.e., DNA extraction) of samples (specimens and controls).
B. PCR assay setup using the sample eluates and an IDH1 amplification reagent kit.
C. Amplification/detection on a real-time PCR instrument.

### Assay Results

For each patient sample, 2 PCR reactions are evaluated. The IDH1 Assay of this Example is a qualitative assay for which specimen interpretations are reported as “Mutation Detected” or “Not Detected.” For specimens with interpretations of “Mutation Detected”, the identity of the IDH1 mutation detected is reported.

### Prevention of Nucleic Acid Contamination

The possibility of nucleic acid contamination is minimized because:
IDH1 Assay of this Example performs amplification and fluorescence detection in a sealed 96-well optical reaction plate.
Detection is carried out automatically without the need to open the 96-well optical reaction plate.
Aerosol barrier pipette tips are used for all pipetting. The pipette tips are discarded after use.
Separate dedicated areas are used to perform IDH1 Assay of this Example.

1. A method of treating a subject having acute myeloid leukemia harboring an isocitrate dehydrogenase-1 (IDH1) mutation as detected by an FDA-approved test, comprising administering to the subject 300 mg of olutasidenib per day in combination with cytarabine.

2. The method of claim 1, wherein the olutasidenib is administered once daily.

3. The method of claim 1, wherein the olutasidenib is administered twice daily.

4. The method of claim 1, wherein the olutasidenib is orally administered to the patient.

5. The method of claim 1, wherein the olutasidenib is administered in the form of a Type A solid form.

6. The method of claim 1, wherein the olutasidenib is administered in a 150 mg strength unit dosage form.

7. The method of claim 1, wherein the olutasidenib is administered for a minimum of 6 months.

8. The method of claim 1, wherein the IDH1 mutation is a R132X mIDH-1 mutation.

9. The method of claim 1, wherein the IDH1 mutation is selected from the group consisting of R132C, R132H, R132S, R132G, and R132L.

10. The method of claim 1, further comprising administering azacitidine to the subject.

11. The method of claim 1, wherein the acute myeloid leukemia is relapsed.

12. The method of claim 1, wherein the acute myeloid leukemia is refractory.

13. A method, comprising identifying a subject with acute myeloid leukemia harboring an isocitrate dehydrogenase-1 (IDH1) mutation, and treating the subject with 300 mg of olutasidenib per day in combination with cytarabine.

14. The method of claim 13, wherein the olutasidenib is administered once daily.

15. The method of claim 13, wherein the olutasidenib is administered twice daily.

16. The method of claim 13, wherein the olutasidenib is orally administered to the patient.

17. The method of claim 13, wherein the olutasidenib is administered in the form of a Type A solid form.

18. The method of claim 13, wherein the olutasidenib is administered in a 150 mg strength unit dosage form.

19. The method of claim 1, wherein the olutasidenib is administered in an amount of 150 mg twice daily.

20. The method of claim 1, wherein cytarabine is administered at a dose of 20 mg twice daily subcutaneously for 10 days in a 28-day cycle.

21. The method of claim 13, wherein the olutasidenib is administered in an amount of 150 mg twice daily.

22. The method of claim 13, wherein cytarabine is administered at a dose of 20 mg twice daily subcutaneously for 10 days in a 28-day cycle.
